(19) United States
(12) Patent Application Publication
(10) Pub. No.: US 2024/0197989 A1
(43) Pub. Date: Jun. 20, 2024

(54) SYSTEMS FOR OUT-PATIENT TREATMENT OF A PATIENT, AND RELATED METHODS

(71) Applicants: AMGEN INC., Thousand Oaks, CA (US); AMGEN RESEARCH MUNICH GMBH, Munich (DE)

(72) Inventors: Paul Gordon, Newbury Park, CA (US); Gerhard Zugmaier, München (DE); Muhammad Faraz Zaman, Simi Valley, CA (US)

(21) Appl. No.: 18/287,046

(22) PCT Filed: Apr. 27, 2022
(86) PCT No.: PCT/US22/26426

§ 371 Date: Oct. 16, 2023

Related U.S. Application Data
(60) Provisional application No. 63/181,002, filed on Apr. 28, 2021.

(51) Int. Cl. A61M 5/172; A61M 5/142; G16H 20/17; G16H 40/67; G16H 50/30; G16H 80/00

(52) U.S. Cl. CPC A61M 5/1723; A61M 5/14248; G16H 20/17; G16H 40/67; G16H 50/30; G16H 80/00; A61M 2205/18; A61M 2205/3303; A61M 2205/3553

(57) ABSTRACT

A system for out-patient treatment with immunotherapy that activates T cells of a patient to fight cancer is provided that may include an infusion pump; a patient wearable device configured to acquire sensor data related to detection of cytokines released by the patient in response to the patient receiving the immunotherapy that activates T cells; and remote wireless communication with a healthcare provider based on the acquired sensor data. A computer-readable medium having computer-readable instructions stored thereon that, when executed by a processor, cause the processor to monitor health of a patient may include a sensor data receiving module that, when executed by a processor, causes the processor to receive sensor data; and a warning data generation module that, when executed by a processor, causes the processor to generate at least one warning based on the sensor data, wherein a content of the sensor data is based on an out-patient treatment that includes an immunotherapy that activates T cells of the patient.

### CROSS-REFERENCE TO RELATED APPLICATION

Priority is claimed to U.S. Patent Application No. 63/181,002, filed Apr. 28, 2021, the entire contents of which are hereby incorporated by reference herein.

### FIELD OF DISCLOSURE

The present disclosure relates to systems and methods to integrate an out-patient site with a healthcare provider site using remote wireless communication with a health care provider (HCP).

### BACKGROUND

Healthcare is undergoing a paradigm shift from reactive, in-hospital treatment to proactive, community-based care. Today's healthcare systems must deliver improved patient outcomes while reducing costs. Hospital overcapacity and the pressure to deliver patient care in the community is driving pent-up demand for care at home. Until recently, health care systems have lacked the technology to satisfy this demand. Various aspects of telemedicine are becoming more common. However, the Health Insurance Portability and Accountability Act (HIPPA), among other health care related laws and rules, place limits on patient-specific health information access, transmission, storage, etc.

As patient desire for out-patient care increases, the need for remote patient health monitoring apparatuses, systems, and methods increases. This increased need for remote patient health monitoring is further increased due to a desire for improved patient outcomes, particularly with regard to higher risk patients (e.g., complete out-patient treatment of patients having residual amounts of leukemia cells in their system, a likelihood of experiencing treatment-emergent adverse events (TEAE), such as, for example, cytokine release syndrome (CRS) and/or neurotoxicity (NT), a likelihood of experiencing other serious adverse events (SAEs) requiring hospitalization during the mandatory device monitoring period (MDMP), etc.).

Apparatuses, systems, and methods are needed for improving out-patient healthcare outcomes. Apparatuses, systems, and methods are also needed for improving out-patient healthcare having improved communication between a patient and associated healthcare provider(s).

### SUMMARY

A system for out-patient treatment may include an infusion pump for delivering a therapeutic to a patient. The system may also include a wearable device configured to be worn by the patient before, during, and/after delivery of the therapeutic. The wearable device may include one or more sensors configured to acquire sensor data related to detection of patient vital signs. The system may further include a wireless communication module disposed on the wearable device and configured for remote wireless communication with a healthcare provider based on the acquired sensor data.

In another embodiment, a computer-readable medium may include computer-readable instructions stored thereon that, when executed by a processor, may cause the processor to monitor health of a patient. The computer-readable medium may include a sensor data receiving module that, when executed by a processor, may cause the processor to receive sensor data. The computer-readable medium may also include a warning data generation module that, when executed by a processor, may cause the processor to generate at least one warning based on the sensor data. A content of the sensor data may be based on an out-patient treatment that includes an immunotherapy that activates T cells of the patient.

In a further embodiment, a computer-implemented method for out-patient treatment with immunotherapy that activates T cells of a patient to kill cancer cells may include receiving, at a processor, sensor data in response to the processor executing a sensor data receiving module. The method may also include generating, using a processor, warning data, based on the sensor data, in response to the processor executing a warning data generation module. A content of the sensor data may be based on a likelihood that the immunotherapy will trigger increased risk factors of cytokine release syndrome.

### BRIEF DESCRIPTION OF THE DRAWINGS

It is believed that the disclosure will be more fully understood from the following description taken in conjunction with the accompanying drawings. Some of the drawings may have been simplified by the omission of selected elements for the purpose of more clearly showing other elements. Such omissions of elements in some drawings are not necessarily indicated of the presence or absence of particular elements in any of the exemplary embodiments, except as may be explicitly delineated in the corresponding written description. Also, none of the drawings are necessarily to scale.

FIG. 1 depicts an example out-patient treatment system;

FIG. 2A depicts a high-level block diagram of an example out-patient treatment system;

FIG. 2B depicts a block diagram of an example sensor device;

FIG. 2C depicts an example method of implementing a sensor device;

FIG. 2D depicts a block diagram of an example patient wearable device;

FIG. 2E depicts an example method of implementing a patient wearable device;

FIG. 2F depicts a block diagram of an example patient device;

FIG. 2G depicts an example method of implementing a patient device;

FIG. 2H depicts a block diagram of an example healthcare provider device;

FIG. 2J depicts an example method of implementing a healthcare provider device;

FIG. 2K depicts a block diagram of an example server;

FIG. 2L depicts an example method of implementing a server;

FIG. 2M depicts a high-level block diagram of an example out-patient treatment system;

FIG. 2N depicts a high-level block diagram of an example out-patient treatment system;

FIG. 3A depicts an example patient wearable device;

FIG. 3B depicts an example patient wearable device;

FIG. 3C depicts an example patient wearable device;

FIG. 4A depicts an example sensor device;

FIG. 4B depicts an example sensor device;

FIG. 4C depicts an example sensor device;

FIG. 4D depicts an example sensor device;

FIGS. 5A-H and J-L depict various example user interface displays of an out-patient treatment system;

FIGS. 6A and 6B depict example protocol for a phase 4 study; and

FIG. 6C depicts various example monitoring requirements for an out-patient treatment system.

Skilled artisans will appreciate that elements in the figures are illustrated for simplicity and clarity and have not necessarily been drawn to scale. For example, the dimensions and/or relative positioning of some of the elements in the figures may be exaggerated relative to other elements to help to improve understanding of various embodiments of the present invention. Also, common but well-understood elements that are useful or necessary in a commercial feasible embodiment are often not depicted in order to facilitate a less obstructed view of these various embodiments. It will further be appreciated that certain actions and/or steps may be described or depicted in a particular order of occurrence while those skilled in the art will understand that such specificity with respect to sequence is not actually required. It will further be appreciated that certain actions and/or steps may be described or depicted in a particular order of occurrence while those skilled in the art will understand that such specificity with respect to sequence is not actually required. It will also be understood that the terms and expressions used herein have the ordinary technical meaning as is accorded to such terms and expressions by persons skilled in the technical field as set forth above except where different specific meanings have otherwise been set forth herein.

### DETAILED DESCRIPTION

Reliable, continuous patient monitoring, as described herein, may enable early intervention in the event of patient deterioration, a better experience for patients, fewer unnecessary hospital re-admissions, customers have been able to shorten hospital stays, reduce hospital readmission rates, and deliver better patient outcomes. Apparatuses, systems, and methods are provided for improving out-patient healthcare outcomes. Apparatuses, systems, and methods are also provided for improving out-patient healthcare having improved communication between a patient and associated healthcare provider(s). In a specific embodiment (described herein as an “EXAMPLE STUDY”), a system is provided for out-patient treatment with immunotherapy that activates T cells of a patient to fight cancer. The system may include an infusion pump (e.g., blinatumomab infusion, Blincyto® as described in international patent application WO2020/221792, the entire content of the disclosure of which is incorporated herein by reference). In another embodiment, a system may be provided for out-patient treatment with acapatamab (AMG 160) as described in international patent application WO 2017/134158, the entire content of the disclosure of which is incorporated herein by reference, a product that contains a half-life extended (HLE) anti-prostate-specific membrane antigen (PSMA)×anti-CD3 BiTE® (bispecific T cell engager), etc.). As described in more detail herein,

In any event, a patient wearable device configured to acquire sensor data (e.g., patient vital signs data, respiratory rate data, heart rate data, oxygen data, blood pressure data, temperature data, patient motion data, patient orientation data, etc.) related to detection of symptoms that may result from the administered medication (e.g., cytokines released by the patient in response to the patient receiving immunotherapy that activates T cells, etc.). The system may also include remote wireless communication between the patient and a healthcare provider based on the acquired sensor data.

As described in detail herein, apparatuses, systems, and methods are provided that may acquire sensor data based on a type of therapy being administered (e.g., based on a type of medication; for AMG 160, a wearable device may monitor patient vital signs for 3 days after a 60 min infusion, etc.) to a respective patient. As also described in detail herein, apparatuses, systems, and methods are provided that generate warning data based on the acquired sensor data. As further described in detail herein, apparatuses, systems, and methods are provided that facilitate audio/video communication between a patient and a respective healthcare provider, along with transmission of sensor data and warning data to a healthcare provider.

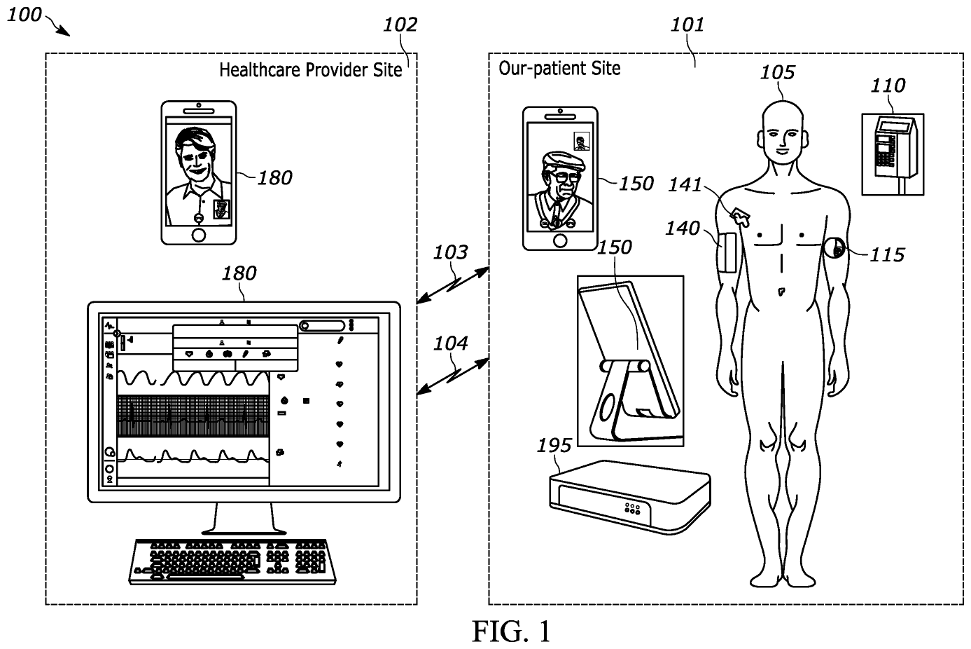

Turning to FIG. 1, an out-patient treatment system 100 may integrate an out-patient site 101 with a healthcare provider site 102. Details of a specific implementation of an out-patient treatment system 100 are included herein with reference to an EXAMPLE STUDY (i.e., a phase 4 feasibility study that evaluated outpatient blinatumomab in subjects (patients 105) with MRD of B-precursor ALL). Other implementations of the out-patient treatment system 100 may be directed to, for example, ongoing, out-patient treatments that benefit from and/or require remote access to vital signs of the patient (i.e., broadly referred to herein as “sensor data”), near real-time remote alarms that are based on the sensor data, and frequent audio/video interaction between a healthcare provider 106 and the patient 105.

With further reference to FIG. 1, the patient 105 may be located at the out-patient site 101 (e.g., the patient's home, a caretaker's home, etc.). The patient 105 may receive an out-patient treatment (e.g., an out-patient treatment that includes an immunotherapy that activates T cells of the patient to fight cancer, an out-patient treatment that may cause a patient to release cytokines, an out-patient treatment that requires a monitoring system, etc.) via, for example, an infusion pump 110. While the treatment may include an infusion pump, any given treatment may not include an infusion pump (e.g., an oral ingestion, injection via a syringe, etc.). When an infusion pump is included, infusion pump data may be acquired from and transmitted to, for example, a controller of the infusion pump, and may be communicatively and functionally integrated into the out-patient treatment system 100.

The out-patient treatment system 100 may also include a patient wearable device 115 (e.g., a wearable device as available from Current Health device (https://currenthealth.com/), a wearable device as available from Bio Beat device (https://www.bio-beat.com/), or any suitable/required remote and/or wearable monitor, etc.). The patient wearable device 115 may include a lithium ion battery with, for example, a battery life of 36 hours. The battery may be charged using, for example, wireless Qi induction charging, with a charge time of 3 hours. While not shown in FIG. 1, the out-patient treatment system 100 may also include a wireless Qi induction charger that has a mold to enable placement of the CH device sensor. The charger may be plugged into a standard home power outlet. It requires no configuration and has no buttons.

A patient wearable device assembly may be provided to a patient 105 that includes, for example, at least three components: 1) reusable cradle, 2) a fabric single-use strap (in 5 sizes), and 3) a reusable sensor. The sensor may be inserted into the cradle, and may power on through detection of magnets in the cradle. The strap may be inserted into the cradle on both sides. The patient wearable device 115 may be, for example, slid up an arm of the patient like a tourniquet, and then tightened such that the patient wearable device 115 closely moves with the skin. The reusable sensor may include a battery having a battery life of, for example, 36 hours, and the patient 105, may be instructed to swap the sensor every 24 hours, 1 on the charger, and 1 on their arm. The reusable sensor may be charged wirelessly by placing the reusable sensor on top of the charging plate.

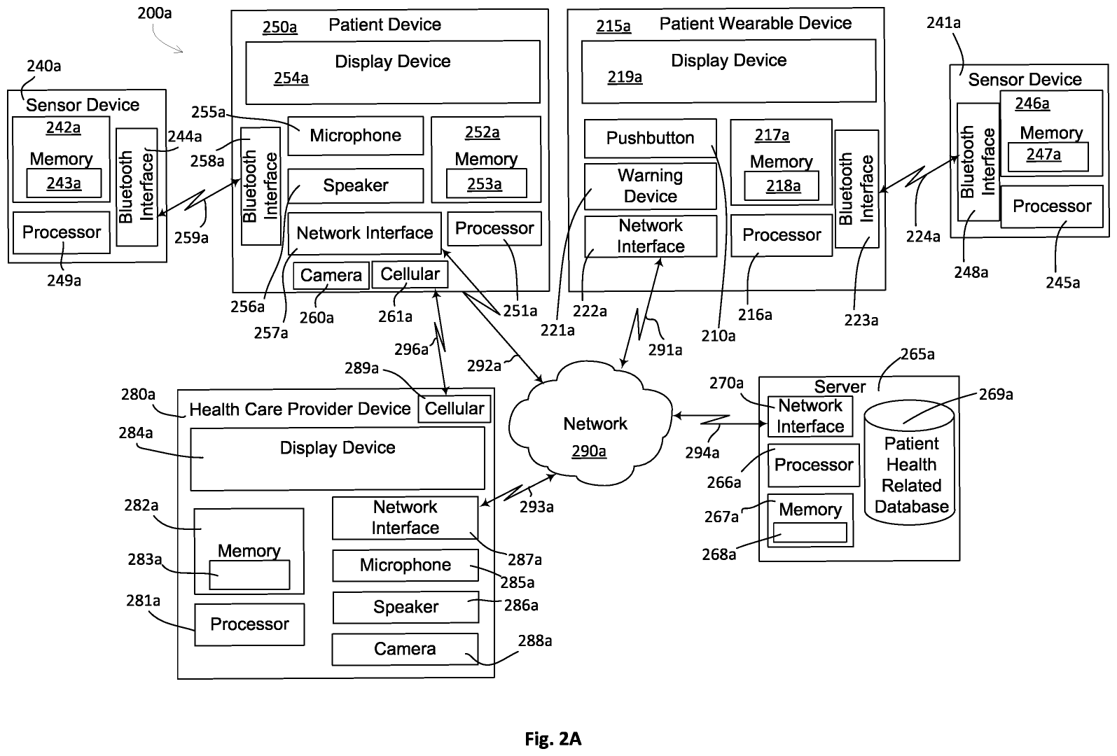

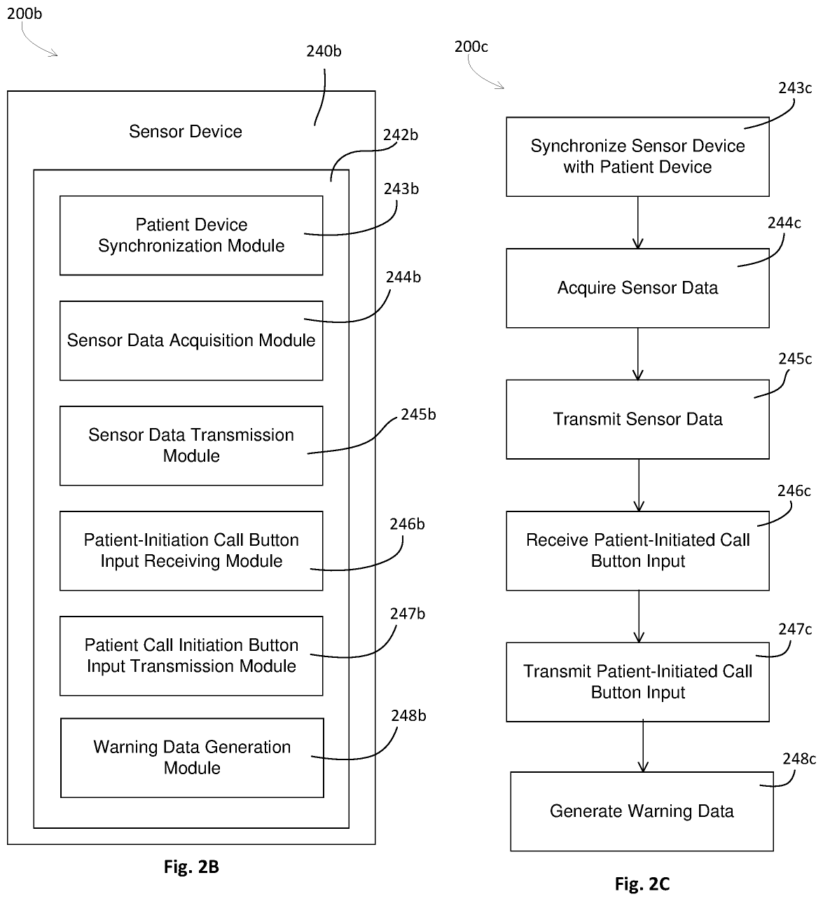

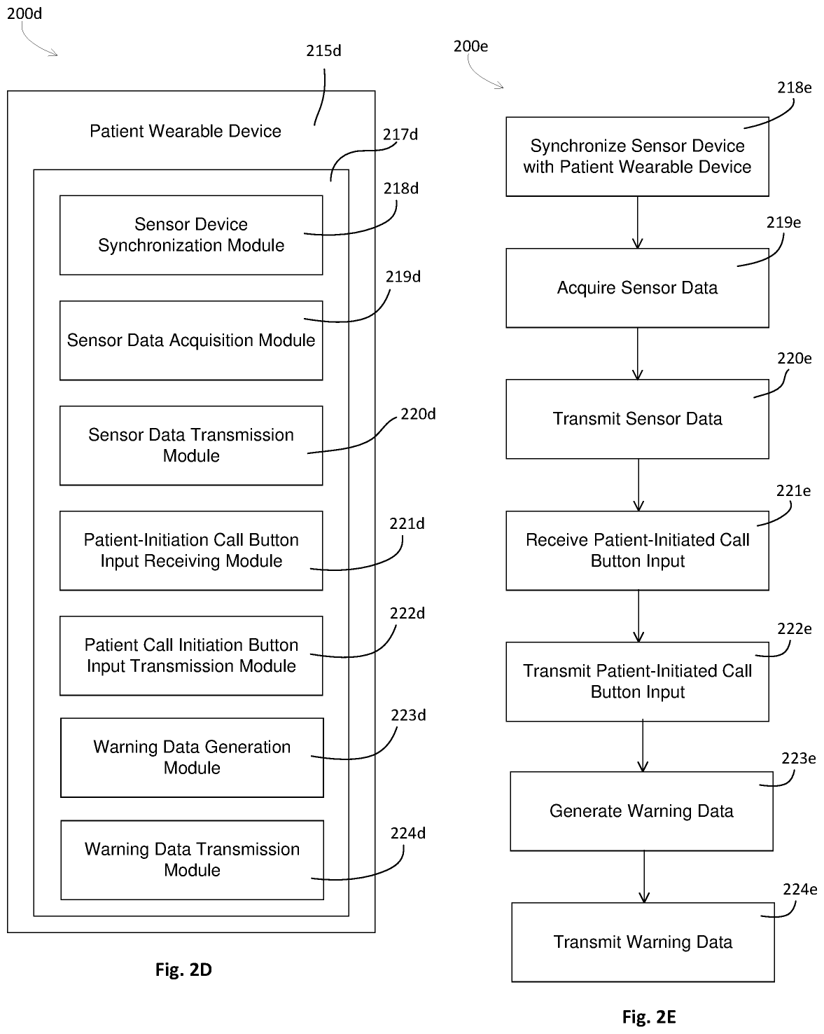

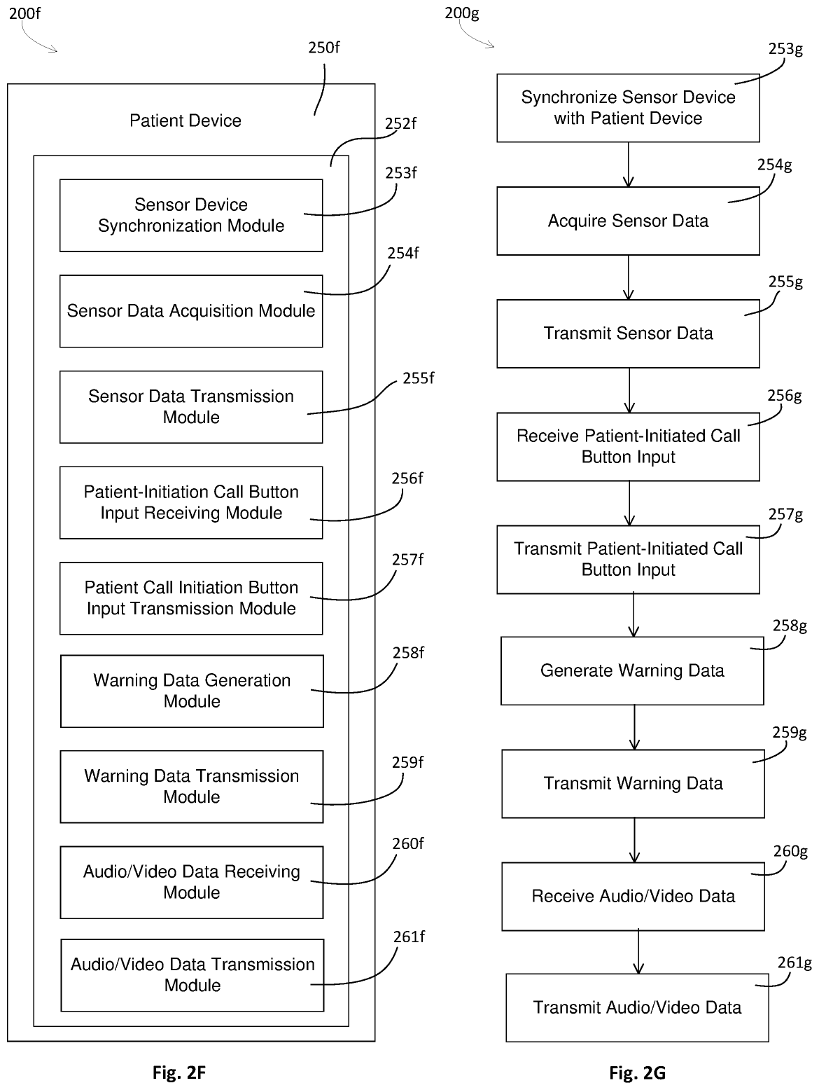

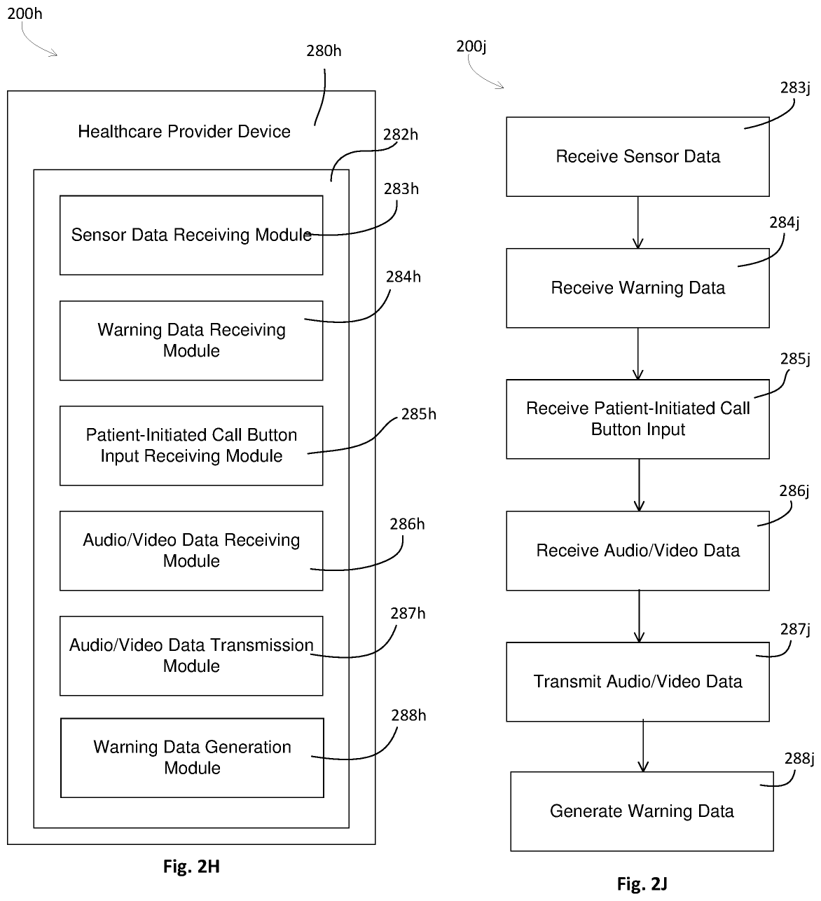

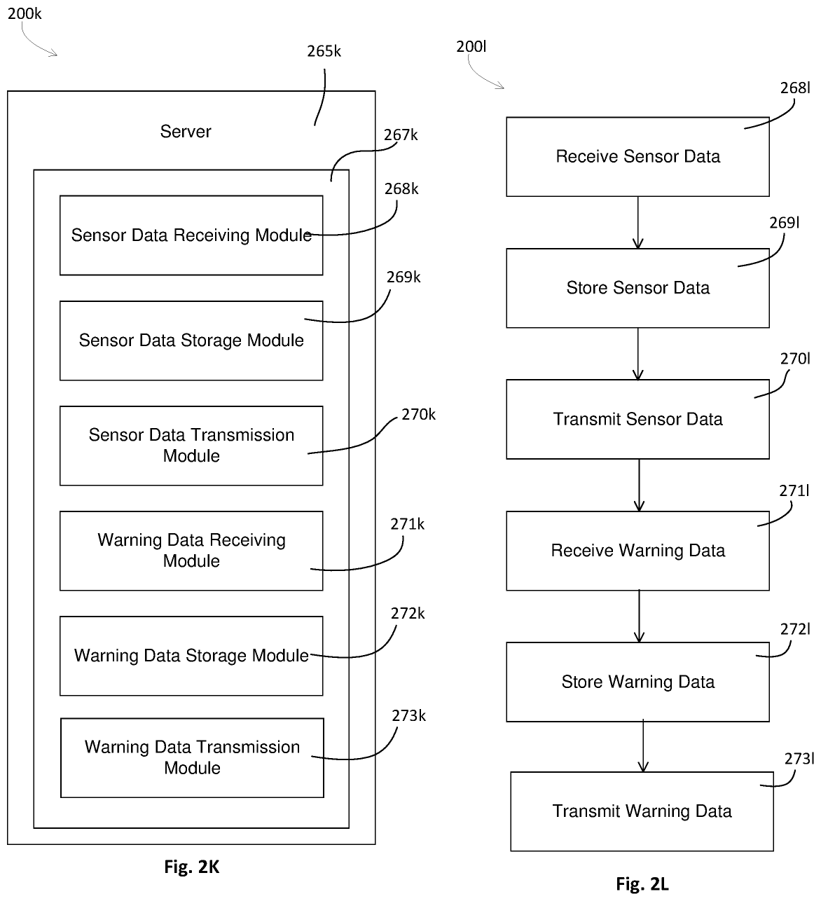

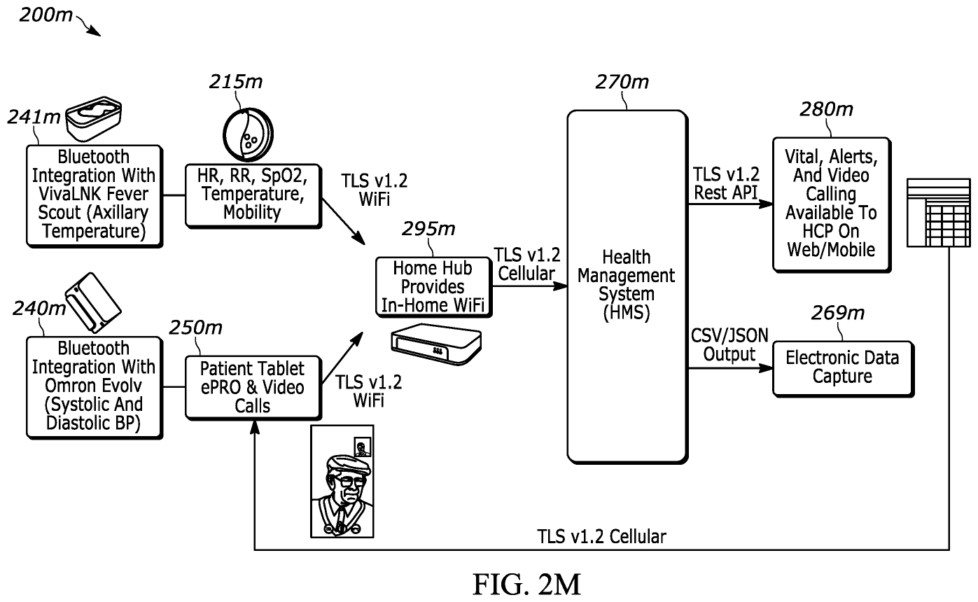

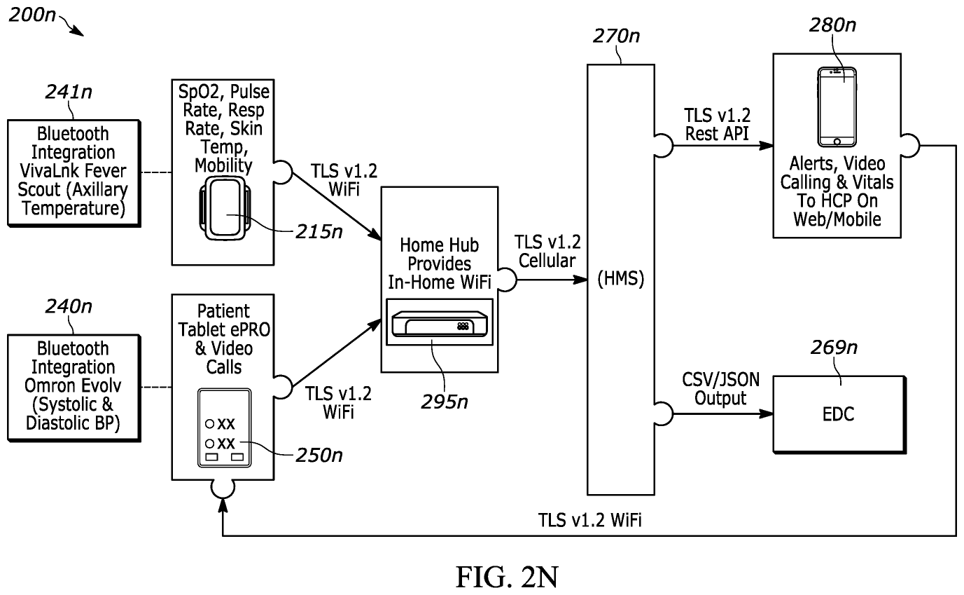

The out-patient treatment system 100 may also include a blood pressure monitor 140 (e.g., an Evolv blood pressure monitor as available from Omron, etc.). The blood pressure monitor 140 may be, for example, an upper-arm worn wireless device that uses an optical sensor, thermistor, accelerometer and gyroscope to measure a subject's heart rate (HR), respiratory rate (RR), axillary temperature, and oxygen saturation. Associated “vital signs” may be measured, for example, every 2 seconds and transmitted every 30 seconds over WiFi. The BP monitor 140 may be, for example, a brachial or radial artery oscillometric BP monitor. It is self-applied by the subject like a tourniquet and, after pressing the start button, will transmit the subject's systolic and diastolic BP over Bluetooth to the subject “tablet app” (e.g., module 253a of FIG. 2A, etc.) as described herein.

The out-patient treatment system 100 may also include an auxiliary temperature sensor 141 (e.g., a Fever Scout auxiliary temperature sensor as available from VivaLNK, etc.). The axillary temperature sensor 141 may be attached to the patient 105 via, for example, an adhesive to the axilla of the patient. An auxiliary temperature sensor 141 may be attached to the patient's axilla for, for example, a duration of an associated mandatory monitoring period. The axillary temperature sensor 141 may, for example, measure axillary temperature of the patient 105 continuously, and may broadcasts the data, via Bluetooth, to the patient wearable device 115. The axillary temperature sensor 141 may be, for example, a single subject use, and may include a battery having a battery life that is longer than the mandatory device monitoring period. As described herein, an auxiliary temperature sensor 141 may be attached to the patient's axilla for, for example, a duration of an associated mandatory monitoring period (e.g., days, weeks, months, etc.).

While the patient wearable device 115, the blood pressure monitor 140, and the auxiliary temperature sensor 141 are shown in FIG. 1 as being separate device, the patient wearable device 115, the blood pressure monitor 140, the auxiliary temperature sensor 141, or any sub-combination thereof may be integrated within a single device (e.g., a single patient wearable device). Additionally, any given patient wearable device 115 may include other sensor(s) (e.g., a heart rate sensor, an oxygen sensor, etc.). As described in more detail elsewhere herein, any given patient wearable device may include an “I do not feel well button” that may enable the patient 105 to initiate a warning and/or initiate an audio and/or video conversation with a healthcare provider and/or emergency services.

The out-patient treatment system 100 may also include a patient device 150 (e.g., a tablet computer w/ePro software, a smart phone, etc.) and a network device 195 (e.g., a home hub, etc.). The network device 195 may, for example, provide WiFi signal within a subject home and transmits data out over the cellular network to CH's software platform. The network device 195 may be, for example, plugged into a standard home power outlet. The network device 195 may not require any patient configuration. Similarly, the network device 195 may not include any buttons (e.g., patient inputs, etc.). The patient 105 may plug the network device 195 into a standard home 110V power outlet. While not shown in FIG. 1, the network device 195 may include at least three solid LEDs configured to, for example, indicate that the network device 195 is powered on and transmitting. The patient device 150 and the network device 195 may be, for example, configured to support remote wireless communication (e.g., sensor data communication, audio communication, video communication, etc.) with a healthcare provider based on the acquired sensor data and/or warning data.

As described in detail herein, a user interface displayed on, for example, a display (e.g., display 254a of FIG. 2A, etc.) of a patient device 150 may allow the patient 105 to select a “BP reminder,” which may guide the patient 105 through a process to collect their own BP. Notably, the patient 105 is typically seated, and relaxed. The BP cuff 140, for example, may be slid up an arm of the patient 105 like a tourniquet, and then tightened. The patient 105 typically relaxes their arm. As described in detail herein, the patient 105 may press a start button (not shown in FIG. 1) on the BP monitoring device 140. As further described in detail, the collected BP may be, for example, displayed on the patient device 150, and transmitted to, for example, the server 265a of FIG. 2A.

The out-patient treatment system 100 may also include a “Software Platform” (e.g., a combination of modules 253a and 268a of FIG. 2A, etc.). The software platform may be, for example, at least in part be hosted on Amazon Web Services (AWS), within a United States (US) data center. As described in detail herein, the software platform 253a, 268a, when executed by one or more processors (e.g., processors 251a, 266a of FIG. 2A, etc.) may, for example, cause the processor(s) 251a, 266a to receive sensor data (e.g., data representative of vital signs of a patient 105, patient wearable device data, blood pressure monitor data, auxiliary temperature sensor data, etc.). As further described in detail herein, the software platform 253a, 268a, when executed by one or more processors (e.g., processors 251a, 266a of FIG. 2A, etc.) may, for example, cause the processor(s) 251a, 266a to generate alerts (e.g., transmission of sensor data, generation of warning data, transmission of warning data, initiation of an audio/video conversation between the patient 105 and the healthcare provider 106, etc.) based on the sensor data. For example, the processor(s) 251a, 266a may generate alerts based on preset alarming thresholds, and then transmit associated warning data to the healthcare provider (HCP) 180.

The module 253a may be configured as a “Subject Tablet App” pre-installed on, for example, the patient device 150. The patient device 150 may be provided to a patient 105 in, for example, a “kiosk mode” (i.e., a mode where module 253a is the only “application” that can be accessed by the patient 105). As described in detail herein, execution of the module 253a by processor 251a may, for example, cause the processor 251a to capture electronic patient-reported outcomes (ePRO) data, capture BP data from the integrated BP monitor 140 and initiate a video call between the patient 105 and the healthcare provider 106.

The software platform 253a, 268a may, for example, further include a “healthcare provider (HCP) App” (e.g., module 283a of FIG. 2A, etc.). The “HCP app” may be provided to the healthcare provider 106 on, for example, an Android phone. Additionally, or alternatively, the “HCP app” may be accessible via an internet browser. As described in detail herein, execution of the module 283a by a processor (e.g., processor 281a of FIG. 1A, etc.) may cause the processor 281a to receive health alerts for the subject(s) monitored, allow monitoring of associated vital signs and conduct video calls to the subject.

As described in detail herein, the out-patient treatment system 100 may include two independent communication links between the patient site 101 and the healthcare provider site 102. A first communication link 103 (e.g., similar to a combination of communication links 290a, 292a, and 293a of FIG. 2A, etc.) may, for example, route data (e.g., sensor data, warning data, patient-initiated call data, etc.) between the patient site 101 and the healthcare provider site 102. A second communication link 104 (e.g., similar to communication link 296a of FIG. 2A, etc.) may, for example, route data (e.g., audio and/or video data, etc.) between the patient site 101 and the healthcare provider site 102.

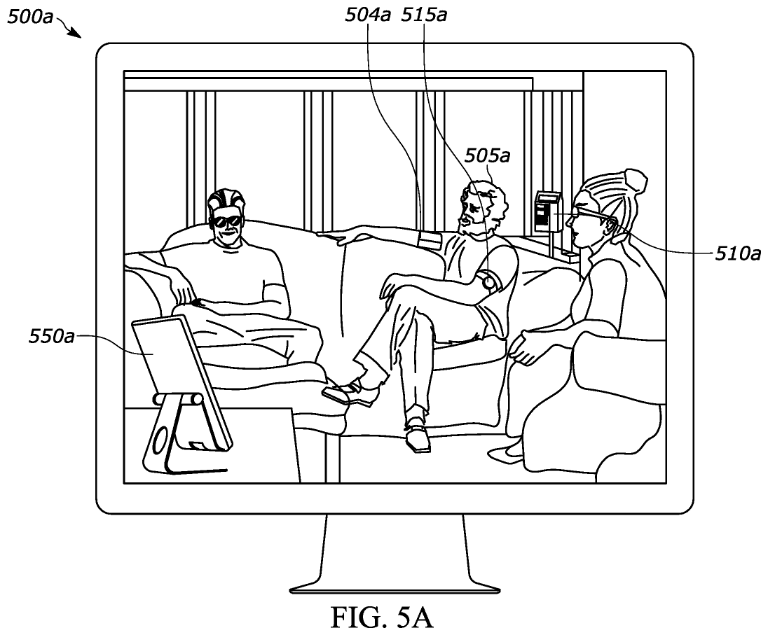

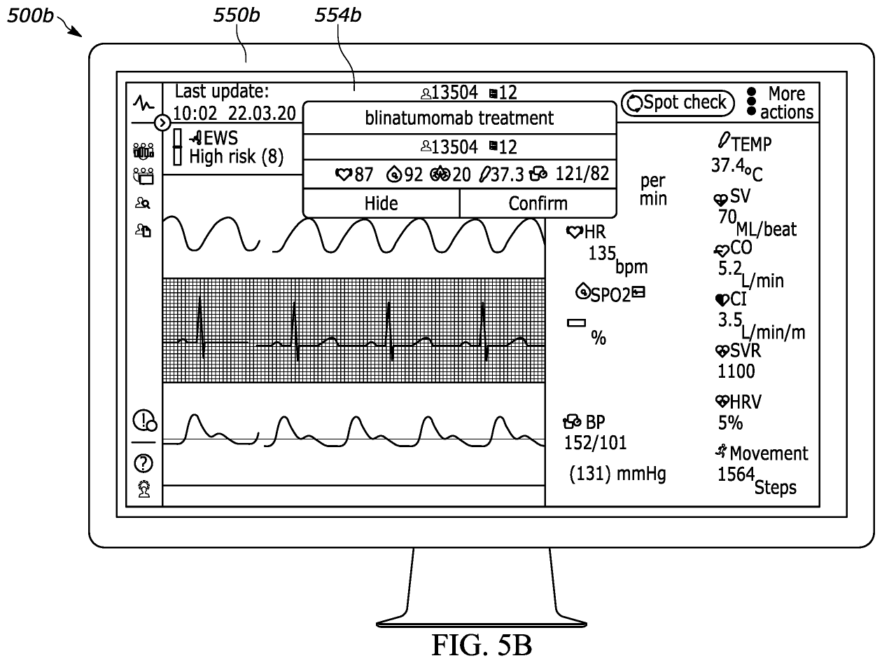

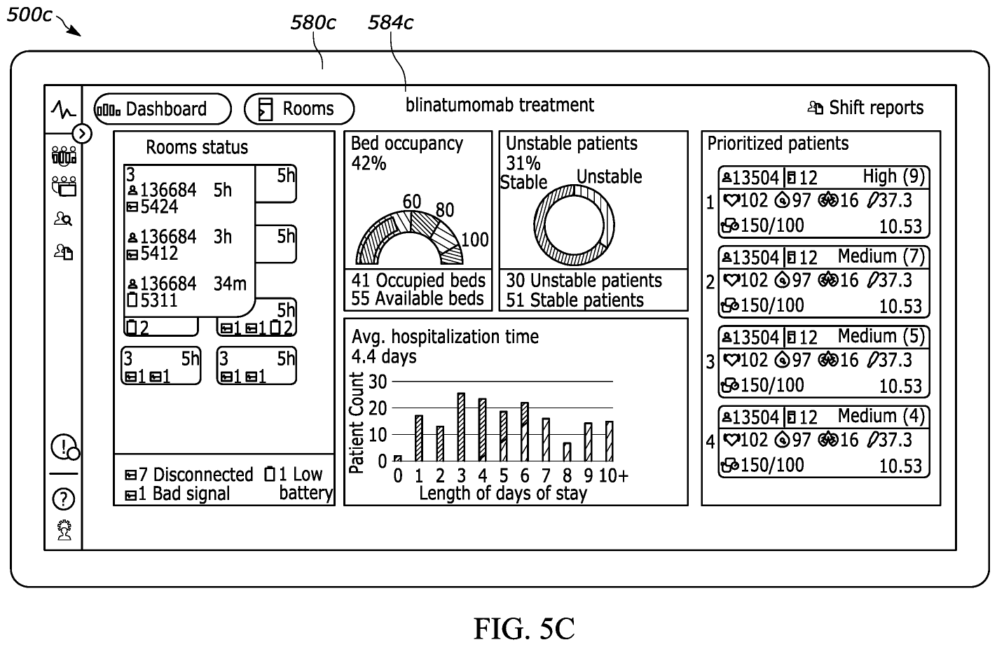

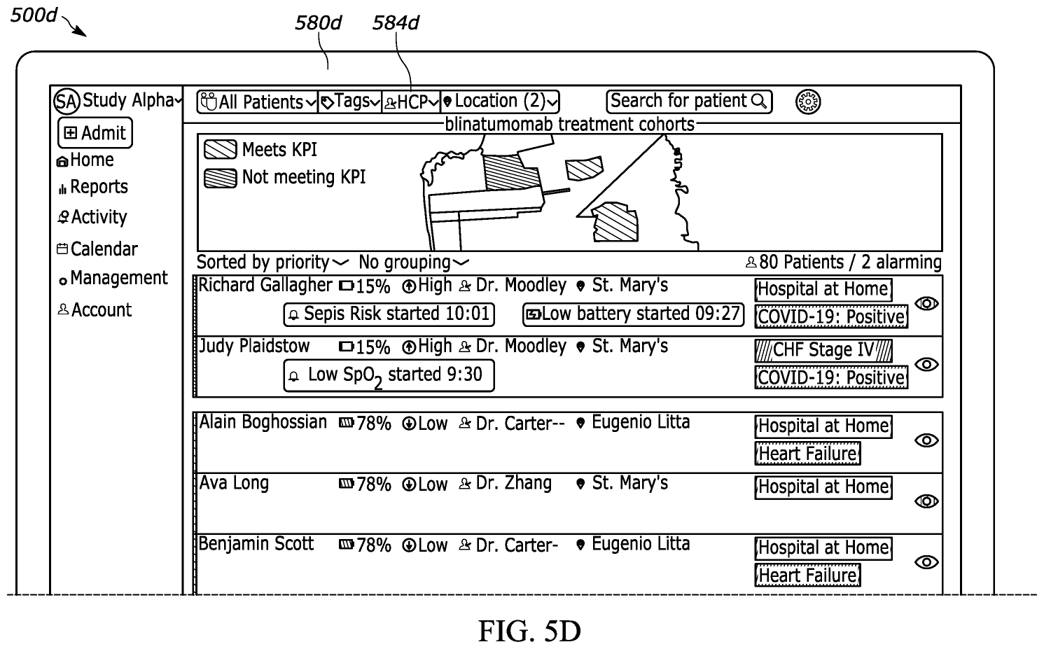

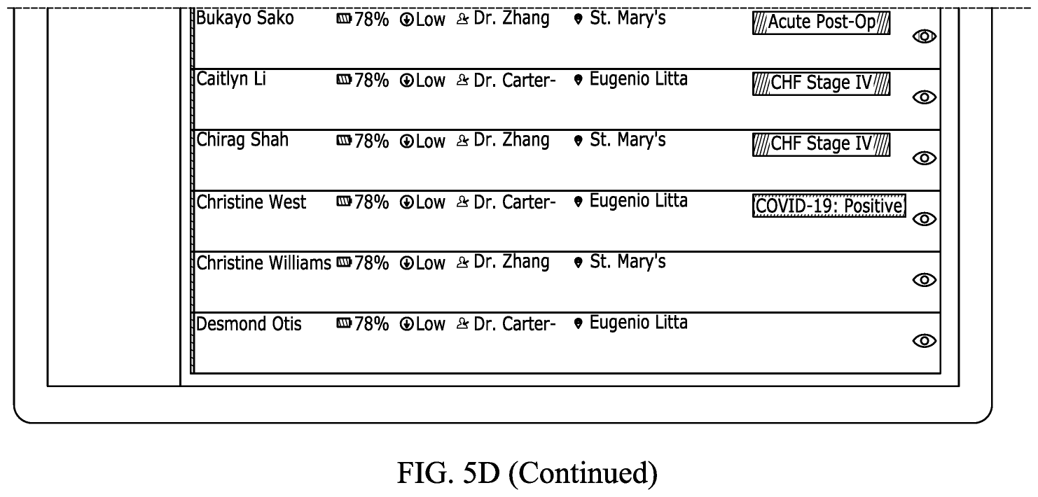

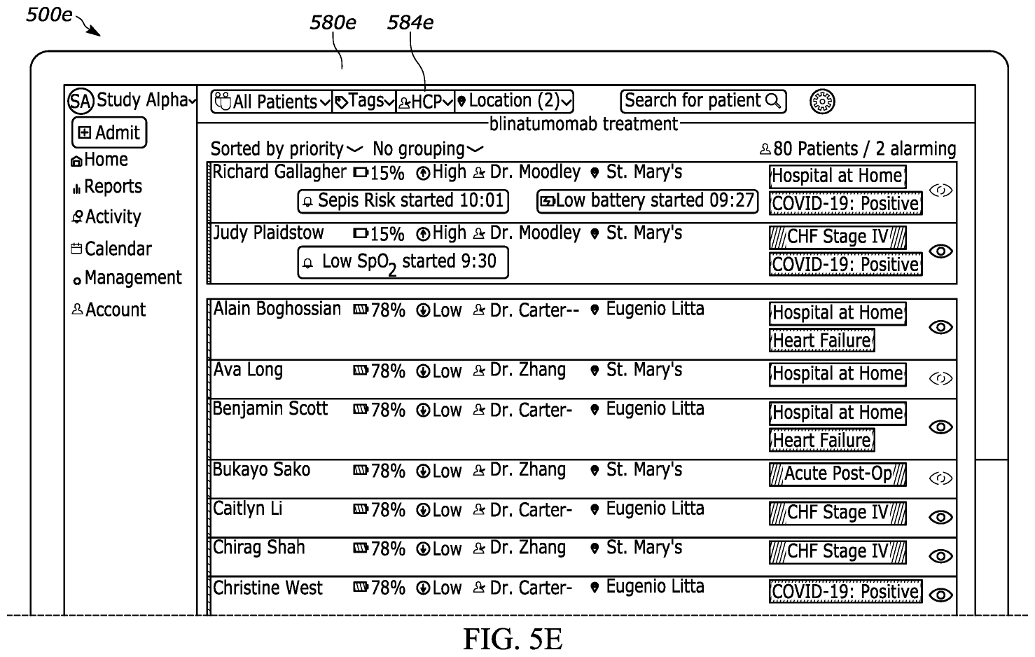

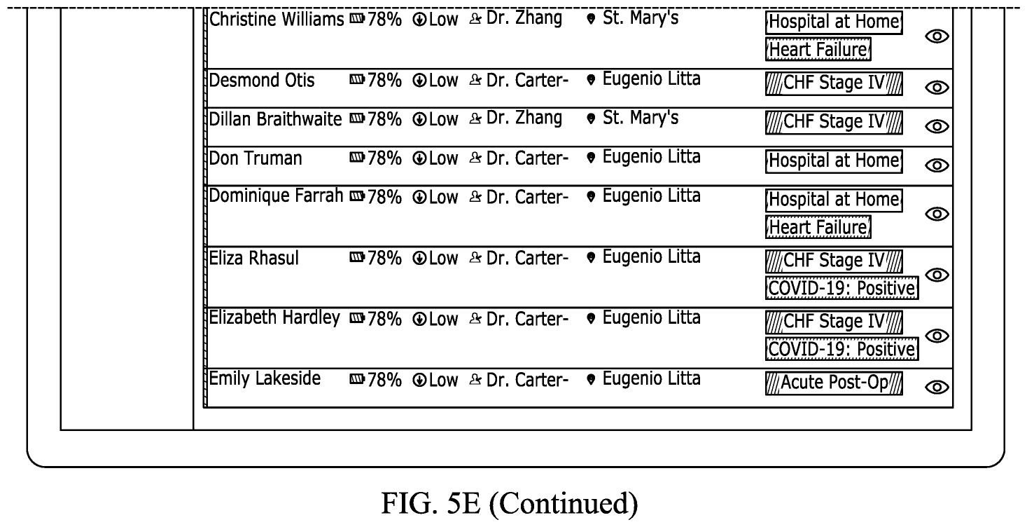

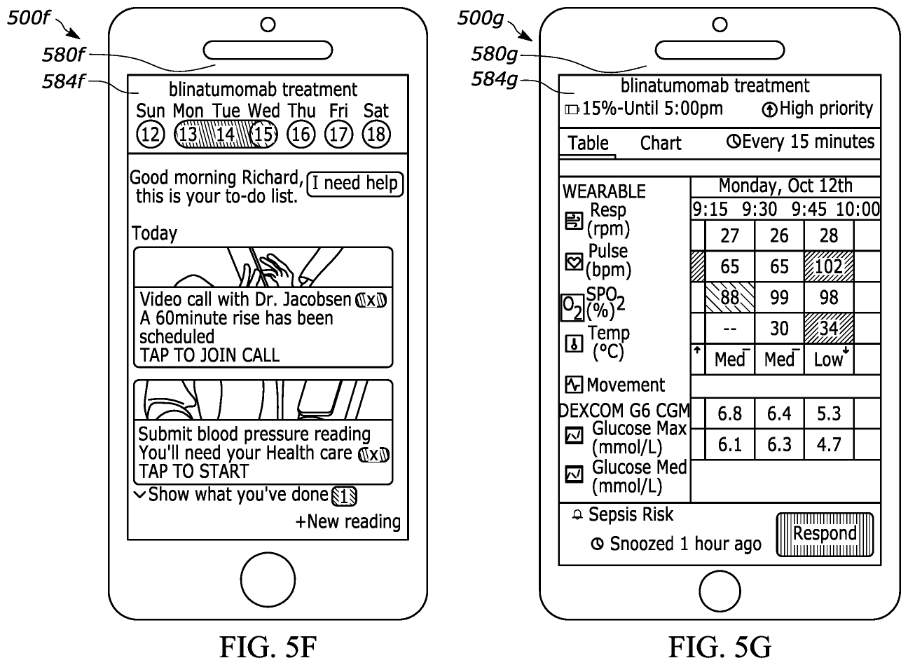

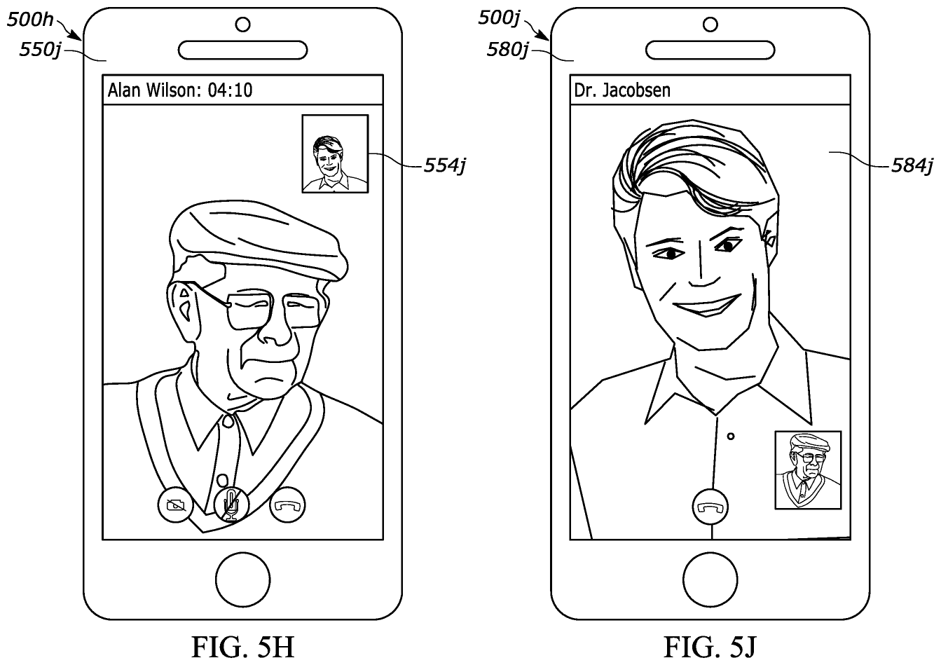

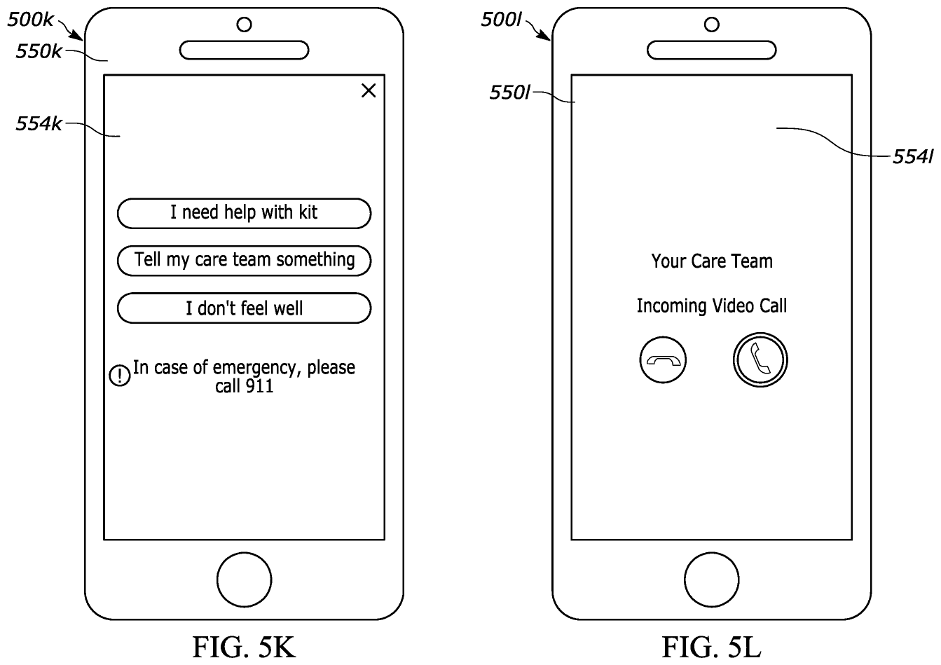

Clinical dashboards (e.g., as illustrated in displays 500a-h,j-l of FIGS. 5A-H and J-L, etc.) may be accessible via web, iOS, and android and integrated with the EMR for easy access. Prioritize urgent cases may include patients that are stratified by risk with role-based permissions and notification routing to help with prioritization. Collaborate with a “Team In-App” where team members can easily share notes on specific patients or alarms to improve collaboration. The systems may allow: access to deep clinical insights; tailor alerts based on clinical pathway; alerts tailored to a clinical pathway and specific patient population to reduce alert burden; move away from one-off data points; alarms based on sustained changes in patient vitals over time, rather than momentary changes; reduce alarm burden, alarms triggered only when signs change simultaneously (e.g., movement low but pulse and resp. rate increasing); leverage machine learning to improve care delivery; customize patient focused algorithms; configurable algorithms defined at individual or population level to measure changes in patient baseline; predict patient deterioration earlier; machine learning models that predict the onset of patient disease and risk of patient hospitalization; accelerate research initiatives; and over 1 million hours of labelled human health data available for research and development of new therapies.

An associated “kit” assembly, may be contained within, for example, a soft case that is easily transportable to the patient's home. A kit assembly may include: at least one infusion pump 110, at least one sensor device 140, 141, at least one patient wearable device 115, at least one patient device 150, and at least one network device 195. While not shown in FIG. 1, a kit assembly may be provided to a patient 105 pre-provisioned and fully configured. Infusion pumps, IV bags and tubing, and ancillary materials (e.g., syringes, sterile needles, alcohol prep pads) may be included in a kit assembly.

With reference to FIG. 2A, a out-patient treatment system 200a may include a patient wearable device 215a, a patient device 250a, a server 260a, and a healthcare provider device 280a communicatively interconnected via a network 290a. The out-patient treatment system 200a may be similar to, for example, the out-patient treatment system 100 of FIG. 1. The patient wearable device 215a may be similar to, for example, the patient wearable device 115. The patient device 250a may be similar to, for example, the patient device 150 of FIG. 1. The healthcare provider device 280a may be similar to, for example, the healthcare provider device 180 of FIG. 1. The network device 250a may be similar to, or communicatively connected to, for example, the network device 195 of FIG. 1.

While not shown in FIG. 2A, the out-patient treatment system 200a may include an infusion pump device. An infusion pump device may include structure and function similar to, for example, a sensor device 240a, 241a, the patient wearable device 215a, the patient device 250a, a combination thereof, or any sub-combination thereof. Additionally, an infusion pump device may generate and transmit data related to operation of a respective infusion pump and/or information related to a respective medication. Furthermore, any one of the sensor devices 240a, 241a, the patient wearable device 215a, the patient device 250a, the server 260a, or the healthcare provider device 280a may transmit data (e.g., infusion pump control data, infusion pump on/off data, infusion pump alarm data, sensor data, warning data, patient-initiated call data, audio/video data, etc.) to an infusion pump device.

As described in detail herein, the server 260a may, for example, host at least a portion of the “Software Platform” (e.g., module 268a, etc.) including interfaces to the “Subject Tablet App” 253a and the “healthcare provider (HCP) App” 283a. Thereby, the server 260a may, for example: acquire sensor data: acquire patient-initiate call input data; generate alarm data based on the sensor data and/or the patient-initiate call input data; receive audio/video data; transmit audio/video data; generate user interface displays based on the sensor data, the patient-initiate call input data, the warning data, and the audio/video data. Additionally, the server 260a may, for example, store the sensor data, the patient-initiate call input data, the warning data, and/or the audio/video data in a patient health related database 269a.

For clarity, only two sensor devices 240a, 241a, one patient wearable device 215a, one patient device 250a, one server 265a, one healthcare provider device 280a, and one network device 290a are depicted in FIG. 2A. While FIG. 2A depicts only two sensor devices 140a, 141a, one patient wearable device 215a, one patient device 250a, one server 265a, one healthcare provider device 280a, and one network device 290a, it should be understood that any number of sensor devices 140a, 141a, patient wearable devices 215a, patient devices 250a, servers 265a, healthcare provider devices 280a, and/or network devices 290a may be supported by the out-patient treatment system 200a.

A patient wearable device 215a may include a memory 217a and a processor 216a for storing and executing, respectively, a module 218a. The module 218a, stored in the memory 217a as a set of computer-readable instructions, may be related to an application for implementing at least a portion of the out-patient treatment system 200a. As described in detail herein, the processor 216a may execute the module 218a to, among other things, cause the processor 216a to receive, generate, and/or transmit data (e.g., sensor data, patient-initiated call data, warning data, audio/video data, etc.) with the sensor device 241a, the network device 290a, the patient device 250a, the server 265a, and/or the healthcare provider device 280a.

The patient wearable device 215a may also include a user interface 219a which may be any type of electronic display device, such as touch screen display, a liquid crystal display (LCD), a light emitting diode (LED) display, a plasma display, a cathode ray tube (CRT) display, or any other type of known or suitable electronic display along with a user input device. A user interface 219a may exhibit a user interface display (e.g., any user interface 500a-h,j-l of FIGS. 5A-H and J-L, etc.) which may, for example, depict a user interface for implementation of at least a portion of the out-patient treatment system 200a.

The patient wearable device 215a may also include a patient-initiated call button 210a, a warning device 221a, a network interface 222a, and a Bluetooth interface 223a. The network interface 222a may be configured to facilitate communications, for example, between the patient wearable device 215a and the network device 290a via any wireless communication network 291a, including for example: a wireless LAN, MAN or WAN, WiFi, TLS v1.2 WiFi, the Internet, or any combination thereof. Moreover, a patient wearable device 215a may be communicatively connected to any other device via any suitable communication system, such as via any publicly available or privately owned communication network, including those that use wireless communication structures, such as wireless communication networks, including for example, wireless LANs and WANs, satellite and cellular telephone communication systems, etc.

The Bluetooth interface 223a may be configured to facilitate communications, for example, between the patient wearable device 215a and the sensor device 241a via any wireless communication network 224a, including for example: a Bluetooth link, a wireless LAN, MAN or WAN, WiFi, TLS v1.2 WiFi, the Internet, or any combination thereof. Additionally, or alternatively, a patient wearable device 215a may be communicatively connected to any other device via any suitable communication system, such as via any publicly available or privately owned communication network, including those that use wireless communication structures, such as wireless communication networks, including for example, wireless LANs and WANs, satellite and cellular telephone communication systems, etc.

The sensor device 241a may be similar to, for example, the sensor device 141 of FIG. 1. The sensor device 241a may include a processor 245a, a memory 246a for storing and executing, respectively, a module 247a. The module 247a, stored in the memory 246a as a set of computer-readable instructions, may be related to an application for implementing at least a portion of the out-patient treatment system 200a. As described in detail herein, the processor 245a may execute the module 247a to, among other things, cause the processor 245a to receive, generate, and/or transmit data (e.g., sensor data, patient-initiated call data, warning data, audio/video data, etc.) with the patient wearable device 215a.

The sensor device 241a may also include a Bluetooth interface 248a. The Bluetooth interface 248a may be configured to facilitate communications, for example, between the sensor device 241a and the patient wearable device 215a via any wireless communication network 224a, including for example: a Bluetooth link, a wireless LAN, MAN or WAN, WiFi, TLS v1.2 WiFi, the Internet, or any combination thereof. Additionally, or alternatively, a sensor device 241a may be communicatively connected to any other device via any suitable communication system, such as via any publicly available or privately owned communication network, including those that use wireless communication structures, such as wireless communication networks, including for example, wireless LANs and WANs, satellite and cellular telephone communication systems, etc.

A patient device 250a may include a memory 252a and a processor 251a for storing and executing, respectively, a module 253a. The module 253a, stored in the memory 252a as a set of computer-readable instructions, may be related to an application for implementing at least a portion of the out-patient treatment system 200a (e.g., a “Subject Tablet App”, etc.). As described in detail herein, the processor 251a may execute the module 253a to, among other things, cause the processor 251a to receive, generate, and/or transmit data (e.g., sensor data, patient-initiated call data, warning data, audio/video data, etc.) with the sensor device 240a, the network device 290a, the patient wearable device 215a, the server 265a, and/or the healthcare provider device 280a.

The patient device 250a may also include a user interface 254a which may be any type of electronic display device, such as touch screen display, a liquid crystal display (LCD), a light emitting diode (LED) display, a plasma display, a cathode ray tube (CRT) display, or any other type of known or suitable electronic display along with a user input device. A user interface 254a may exhibit a user interface display (e.g., any user interface 500a-h,j-l of FIGS. 5A-H and J-L, etc.) which may, for example, depict a user interface for implementation of at least a portion of the out-patient treatment system 200a.

The patient device 250a may also include a microphone 255a, a speaker 256a, a camera 260a, a network interface 257a, a Bluetooth interface 258a, and a cellular interface 289a. The network interface 257a may be configured to facilitate communications (e.g., sensor data, warning data, patient-initiated call data, etc.), for example, between the patient device 250a and the network device 290a via any wireless communication network 292a, including for example: TLS v1.2 WiFi, a wireless LAN, MAN or WAN, WiFi, the Internet, or any combination thereof. Moreover, a patient device 250a may be communicatively connected to any other device via any suitable communication system, such as via any publicly available or privately owned communication network, including those that use wireless communication structures, such as wireless communication networks, including for example, wireless LANs and WANs, satellite and cellular telephone communication systems, etc.

While the patient device 250a is illustrated in FIG. 2A as a single device, it should be understood that a patient device 250a may, for example, include a first patient device 250a configured to remain at a patient site 101, and a second patient device (e.g., a cellular telephone, etc.) configured to facilitate data transfer (e.g., sensor data, patient-initiated call data, warning data, audio data, video data, etc.) between the remaining devices in the out-patient treatment system 200a when the patient leaves the patient site 101 (e.g., when the patient device 250a is no longer in communication range with the network device 295a, etc.).

The Bluetooth interface 258a may be configured to facilitate communications, for example, between the patient device 250a and the sensor device 240a via any wireless communication network 259a, including for example: a Bluetooth link, a wireless LAN, MAN or WAN, WiFi, TLS v1.2 WiFi, the Internet, or any combination thereof. Additionally, or alternatively, a patient wearable device 215a may be communicatively connected to any other device via any suitable communication system, such as via any publicly available or privately owned communication network, including those that use wireless communication structures, such as wireless communication networks, including for example, wireless LANs and WANs, satellite and cellular telephone communication systems, etc.

The cellular interface 261a may be configured to facilitate communications (e.g., audio data, video data, etc.), for example, between the patient device 250a and the healthcare provide device 280a via any wireless communication network 296a, including for example: TLS v1.2 Cellular, a wireless LAN, MAN or WAN, WiFi, TLS v1.2 WiFi, the Internet, or any combination thereof. Moreover, a patient device 250a may be communicatively connected to any other device via any suitable communication system, such as via any publicly available or privately owned communication network, including those that use wireless communication structures, such as wireless communication networks, including for example, wireless LANs and WANs, satellite and cellular telephone communication systems, etc.

The sensor device 240a may be similar to, for example, the sensor device 140 of FIG. 1. The sensor device 240a may include a processor 249a, a memory 242a for storing and executing, respectively, a module 243a. The module 243a, stored in the memory 242a as a set of computer-readable instructions, may be related to an application for implementing at least a portion of the out-patient treatment system 200a. As described in detail herein, the processor 249a may execute the module 243a to, among other things, cause the processor 249a to receive, generate, and/or transmit data (e.g., sensor data, patient-initiated call data, warning data, audio/video data, etc.) with the patient device 250a.

The sensor device 240a may also include a Bluetooth interface 244a. The Bluetooth interface 244a may be configured to facilitate communications, for example, between the sensor device 240a and the patient device 250a via any wireless communication network 259a, including for example: a Bluetooth link, a wireless LAN, MAN or WAN, WiFi, TLS v1.2 WiFi, the Internet, or any combination thereof. Additionally, or alternatively, a sensor device 240a may be communicatively connected to any other device via any suitable communication system, such as via any publicly available or privately owned communication network, including those that use wireless communication structures, such as wireless communication networks, including for example, wireless LANs and WANs, satellite and cellular telephone communication systems, etc.

A server 260a may include a memory 267a and a processor 266a for storing and executing, respectively, a module 268a. The module 268a, stored in the memory 267a as a set of computer-readable instructions, may be related to an application for implementing at least a portion of the out-patient treatment system 200a (e.g., a “Software Platform”, etc.). As described in detail herein, the processor 266a may execute the module 268a to, among other things, cause the processor 266a to receive, generate, and/or transmit data (e.g., sensor data, patient-initiated call data, warning data, audio/video data, etc.) with the network device 290a, the patient device 250a, the patient wearable device 215a, and/or the healthcare provider device 280a.

The server 260a may also include a user interface (not shown in FIG. 2A) which may be any type of electronic display device, such as touch screen display, a liquid crystal display (LCD), a light emitting diode (LED) display, a plasma display, a cathode ray tube (CRT) display, or any other type of known or suitable electronic display along with a user input device. An associated user interface may exhibit a user interface display (e.g., any user interface 500a-h,j-l of FIGS. 5A-H and J-L, etc.) which may, for example, depict a user interface for implementation of at least a portion of the out-patient treatment system 200a.

The server 260a may also include a patient health related database 269a and a network interface 270a. The patient health related database 269a may, for example, store sensor data, patient-initiated call data, warning data, audio/video data, etc. The network interface 270a may be configured to facilitate communications, for example, between the server 260a and the network device 290a via any wireless communication network 294a, including for example: TLS v1.2 Cellular, CSV/JSON Output, TLS v1.2 REST API, a wireless LAN, MAN or WAN, WiFi, TLS v1.2 WiFi, the Internet, or any combination thereof. Moreover, a server 260a may be communicatively connected to any other device via any suitable communication system, such as via any publicly available or privately owned communication network, including those that use wireless communication structures, such as wireless communication networks, including for example, wireless LANs and WANs, satellite and cellular telephone communication systems, etc.

A healthcare provider device 280a may include a memory 282a and a processor 281a for storing and executing, respectively, a module 283a. The module 283a, stored in the memory 282a as a set of computer-readable instructions, may be related to an application for implementing at least a portion of the out-patient treatment system 200a (e.g., a “healthcare provider (HCP) App”, etc.). As described in detail herein, the processor 281a may execute the module 283a to, among other things, cause the processor 281a to receive, generate, and/or transmit data (e.g., sensor data, patient-initiated call data, warning data, audio/video data, etc.) with the network device 290a, the patient wearable device 215a, the patient device 250a, and/or the server 265a.

The healthcare provider device 280a may also include a user interface 284a which may be any type of electronic display device, such as touch screen display, a liquid crystal display (LCD), a light emitting diode (LED) display, a plasma display, a cathode ray tube (CRT) display, or any other type of known or suitable electronic display along with a user input device. A user interface 284a may exhibit a user interface display (e.g., any user interface 500a-h,j-l of FIGS. 5A-H and J-L, etc.) which may, for example, depict a user interface for implementation of at least a portion of the out-patient treatment system 200a.

While the healthcare provider device 280a is illustrated in FIG. 2A as a single device, it should be understood that a healthcare provider device 280a may, for example, include a first healthcare provider device 280a configured to remain at a healthcare provider site 102, and a second healthcare provider device (e.g., a cellular telephone, etc.) configured to facilitate data transfer (e.g., sensor data, patient-initiated call data, warning data, audio data, video data, etc.) between the remaining devices in the out-patient treatment system 200a when the healthcare provider leaves the healthcare provider site 102.

The healthcare provider device 280a may also include a microphone 285a, a speaker 286a, a camera 288a, a network interface 287a, and a cellular interface 289a. The network interface 287a may be configured to facilitate communications (e.g., sensor data, warning data, patient-initiated call data, etc.), for example, between the healthcare provider device 280a and the network device 290a via any wireless communication network 293a, including for example: TLS v1.2 REST API, TLS v1.2 Cellular, CSV/JSON Output, TLS v1.2 REST API, a wireless LAN, MAN or WAN, WiFi, TLS v1.2 WiFi, the Internet, or any combination thereof. Moreover, a healthcare provider device 280a may be communicatively connected to any other device via any suitable communication system, such as via any publicly available or privately owned communication network, including those that use wireless communication structures, such as wireless communication networks, including for example, wireless LANs and WANs, satellite and cellular telephone communication systems, etc.

The cellular interface 289a may be configured to facilitate communications (e.g., audio data, video data, etc.), for example, between the healthcare provide device 280a and the patient device via any wireless communication network 296a, including for example: TLS v1.2 Cellular, a wireless LAN, MAN or WAN, WiFi, TLS v1.2 WiFi, the Internet, or any combination thereof. Moreover, a healthcare provider device 280a may be communicatively connected to any other device via any suitable communication system, such as via any publicly available or privately owned communication network, including those that use wireless communication structures, such as wireless communication networks, including for example, wireless LANs and WANs, satellite and cellular telephone communication systems, etc.

Each of the sensor devices 240a, 241a, the patient wearable device 215a, the patient device 250a, the healthcare provider device 280a, and/or the network device 295a may be, for example, configured such that any given device will store data (e.g., sensor data, patient-initiated call data, warning data, audio data, video data, etc.) within a respective memory 246a, 242a, 217a, 252a, etc. when the given device is unable to communicate data to another device as when fully functional within the out-patient treatment system 200a.

Turning to FIG. 2B, an out-patient treatment system 200b may include a sensor device 200b having a patient device synchronization module, 243b, a sensor data acquisition module 244b, a sensor data transmission module 245b, a patient-initiation call button input receiving module 246b, a patient call initiation button input transmission module 247b, and a warning data generation module 248b, for example, stored on a memory 242b as a set of computer-readable instructions. In any event, the modules 243b-248b may be similar to, for example, the module 243a, 247a of FIG. 2A.

With reference to FIG. 2C, a method of implementing a sensor device 200c may be implemented by a first processor (e.g., either processor 245a, 249a of a respective sensor device 241a, 240a of FIG. 1A) executing, for example, at least a portion of modules 243b-248b of FIG. 2B. In particular, processor 245a may execute the patient device synchronization module 243b to cause the processor 245a to, for example, synchronize a sensor device 241a with the patient wearable device 215a (block 243c). Similarly, processor 249a may execute the patient device synchronization module 243b to cause the processor 249a to, for example, synchronize a sensor device 240a with the patient device 250a (block 243c).

The processor 245a may execute the sensor data acquisition module 244b to cause the processor 245b to, for example, receive sensor data (block 244c). The processor 245a may execute the sensor data transmission module 245b to cause the processor 245b to, for example, transmit sensor data (block 245c). The processor 245a may execute the patient-initiation call button input receiving module 246b to cause the processor 245b to, for example, receive a patient-initiation call button input (block 246c). The processor 245a may execute the patient call initiation button input transmission module 247b to cause the processor 245b to, for example, transmit a patient call initiation button input (block 247c). The processor 245a may execute the warning data generation module 248b to cause the processor 245b to, for example, generate warning data (block 248c). The warning data may be based on, for example, the sensor data and/or the warning data.

Turning to FIG. 2D, an out-patient treatment system 200d may include a patient wearable device 200d having a sensor device synchronization module 218d, a sensor data acquisition module 219d, a sensor data transmission module 220d, a patient-initiation call button input receiving module 221d, a patient call initiation button input transmission module 222d, a warning data generation module 223d, and a warning data transmission module 224d, for example, stored on a memory 217d as a set of computer-readable instructions. In any event, the modules 218d-224d may be similar to, for example, the module 218a of FIG. 2A.

With reference to FIG. 2E, a method of implementing a patient wearable device 200e may be implemented by a processor (e.g., processor 216a of FIG. 1A) executing, for example, at least a portion of modules 218d-224d of FIG. 2D. In particular, processor 216a may execute the sensor device synchronization module 218d to cause the processor 216a to, for example, synchronize a sensor device 241a with the patient wearable device 215a (block 218e).

The processor 216a may execute the sensor data acquisition module 219d to cause the processor 216a to, for example, acquire sensor data from the sensor device 241a (block 219e). The processor 216a may execute the sensor data transmission module 220d to cause the processor 216a to, for example, transmit sensor data (block 220e). The processor 216a may execute the patient-initiation call button input receiving module 221d to cause the processor 216a to, for example, receive a patient-initiation call button input (block 221e). The processor 216a may execute the patient call initiation button input transmission module 222d to cause the processor 216a to, for example, transmit a patient call initiation button input (block 222e). The processor 216a may execute the warning data generation module 223d to cause the processor 216a to, for example, generate warning data (block 223e). The warning data may be, for example, based on the sensor data and/or the patient call initiation button input. The processor 216a may execute the warning data transmission module 224d to cause the processor 216a to, for example, transmit warning data (block 224e).

Turning to FIG. 2F, an out-patient treatment system 200f may include a patient device 200f having a sensor device synchronization module, 253f, a sensor data acquisition module 254f, a sensor data transmission module 255f, a patient-initiation call button input receiving module 256f, a patient call initiation button input transmission module 257f, and a warning data generation module 258f, a warning data transmission module 259f, an audio/video data receiving module 260f, and an audio/video data transmission module 261f, for example, stored on a memory 252f as a set of computer-readable instructions. In any event, the modules 253f-261f may be similar to, for example, the module 253a of FIG. 2A.

With reference to FIG. 2G, a method of implementing a patient device 200g may be implemented by a processor (e.g., processor 251a of FIG. 1A) executing, for example, at least a portion of modules 253f-261f of FIG. 2F. In particular, processor 251a may execute the sensor device synchronization module 253h to cause the processor 251a to, for example, synchronize a sensor device 240a with the patient device 250a (block 253g).

The processor 251a may execute the sensor data acquisition module 254f to cause the processor 251a to, for example, acquire sensor data from sensor device 240a (block 254g). The processor 251a may execute the sensor data transmission module 255f to cause the processor 251a to, for example, transmit sensor data (block 255g). The processor 251a may execute the patient-initiation call button input receiving module 256f to cause the processor 251a to, for example, receive a patient-initiation call button input (block 256g). The processor 251a may execute the patient call initiation button input transmission module 257f to cause the processor 251a to, for example, transmit a patient-initiation call button input (block 257g).

The processor 251a may execute the warning data generation module 258f to cause the processor 251a to, for example, generate warning data (block 258g). The warning data may be, for example, based on the sensor data and the patient-initiation call button input. The processor 251a may execute the warning data transmission module 259f to cause the processor 251a to, for example, transmit warning data (block 259g). The processor 251a may execute the audio/video data receiving module 260f to cause the processor 251a to, for example, receive audio/video data from a healthcare provider device 180, 280a (block 260g). The processor 251a may execute the audio/video data transmission module 261f to cause the processor 251a to, for example, transmit audio/video data to a healthcare provider device 180, 280a (block 261g).

Turning to FIG. 2H, out-patient treatment system 200h may include a healthcare provider device 280h having a sensor data receiving module 283h, a warning data receiving module 284h, a patient-initiated call button input receiving module 285h, an audio/video data receiving module 286h, an audio/video data transmission module 287h, and a warning data generation module 288h, for example, stored on a memory 282h as a set of computer-readable instructions. In any event, the modules 283h-288h may be similar to, for example, the module 283a of FIG. 2A.

With reference to FIG. 2J, a method of implementing a healthcare provider device 200j may be implemented by a processor (e.g., processor 281a of FIG. 1A) executing, for example, at least a portion of modules 283h-288h of FIG. 2H. In particular, processor 281a may execute the sensor data receiving module 283h to cause the processor 281a to, for example, receive sensor data from the patient wearable device 115, 215a and/or the patient device 150, 250a (block 283j).

The processor 281a may execute the warning data receiving module 284h to cause the processor 281a to, for example, receive warning data (block 284j). The processor 281a may execute the patient-initiated call button input receiving module 285h to cause the processor 281a to, for example, receive a patient-initiated call button input (block 285j). The processor 281a may execute the audio/video data receiving module 286h to cause the processor 281a to, for example, receive audio/video data from a patient device 150, 250a (block 286j). The processor 281a may execute the audio/video data transmission module 287h to cause the processor 281a to, for example, transmit audio/video data to the patient device 150, 250a (block 287j). The processor 281a may execute the warning data generation module 288h to cause the processor 281a to, for example, generate warning data (block 288j). The warning data may be based on, for example, the sensor data and/or the patient-initiated call button input.

With reference to Turning to FIG. 2K, out-patient treatment system 200k may include a server 200k having a sensor data receiving module 268k, a sensor data storage module 269k, a sensor data transmission module 270k, a warning data receiving module 271k, a warning data storage module 272k, and a warning data transmission module 273k, for example, stored on a memory 2267k as a set of computer-readable instructions. In any event, the modules 268k-273k may be similar to, for example, the module 268a of FIG. 2A.

With reference to FIG. 2L, a method of implementing a server 200l may be implemented by a processor (e.g., processor 266a of FIG. 1A) executing, for example, at least a portion of modules 268k-273k of FIG. 2K. In particular, processor 266a may execute the sensor data receiving module 268k to cause the processor 266a to, for example, receive sensor data (block 2681).

The processor 266a may execute the sensor data storage module 269k to cause the processor 266a to, for example, store sensor data in the in the patient health related database 269a (block 2691). The processor 266a may execute the sensor data transmission module 270k to cause the processor 266a to, for example, transmit sensor data (block 2701). The processor 266a may execute the warning data receiving module 271k to cause the processor 266a to, for example, receive warning data (block 2711). The processor 266a may execute the warning data storage module 272k to cause the processor 266a to, for example, store warning data in the patient health related database 269a (block 272l). The processor 266a may execute the warning data transmission module 273k to cause the processor 266a to, for example, transmit warning data (block 273l).

Turning to FIG. 2M, an out-patient treatment system 200m may include a first sensor device 241m communicatively connected to a patient wearable device 215m. The out-patient treatment system 200m may also include a second sensor device 240m communicatively coupled to a patient device 250m. The patient wearable device 215m and the patient device 250m may be communicatively connected to the network device 295m. The network device 295m may be communicatively connected to a software platform 270m (e.g., server 265a). The software platform 270m may be communicatively connected to a healthcare provider device 280m and the patient health related database 269m. The out-patient treatment system 200m may be similar to, for example, the out-patient treatment system 100 of FIG. 1 or 200a of FIG. 2A.

With reference to FIG. 2N, an out-patient treatment system 200n may include a first sensor device 241n communicatively connected to a patient wearable device 215n. The out-patient treatment system 200n may also include a second sensor device 240n communicatively coupled to a patient device 250n. The patient wearable device 215n and the patient device 250n may be communicatively connected to the network device 295n. The network device 295n may be communicatively connected to a software platform 270n (e.g., server 265a). The software platform 270n may be communicatively connected to a healthcare provider device 280n and the patient health related database 269n. The out-patient treatment system 200n may be similar to, for example, the out-patient treatment system 100 of FIG. 1, 200a of FIG. 2A, or 200m of FIG. 2M.

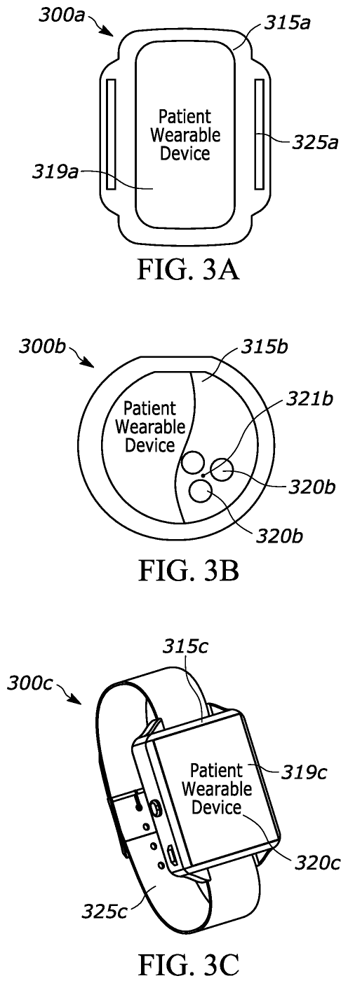

Turning to FIG. 3A, a patient wearable device assembly 300a may include a patient wearable device 315a, a display device 319a, and an arm band 325a. The patient wearable device assembly 300a may be similar to, for example, the patient wearable device 115 of FIG. 1 or 215a of FIG. 2A.

With reference to FIG. 3B, a patient wearable device assembly 300b may include a patient wearable device 315b, a warning indicator 321b, and at least one patient-initiated call button 320b. The patient wearable device assembly 300b may be similar to, for example, the patient wearable device 115 of FIG. 1 or 215a of FIG. 2A.

Turning to FIG. 3C, a patient wearable device assembly 300c may include a patient wearable device 315c, a display device 319c, a patient-initiated call button 320c, and an arm band 325c. The patient wearable device assembly 300c may be similar to, for example, the patient wearable device 115 of FIG. 1 or 215a of FIG. 2A.

A patient wearable device assembly 300a-c may be designed for in-clinic and remote patient monitoring applications. For example, a multi-function cardiac patch may generate live stream multiple parameters to a mobile device or the cloud. A patient wearable device assembly 300a-c may be reusable, rechargeable, and can record data even in the event of a network disruption.

The patient wearable device assembly 300a-c may be powerful cardiac patch has been used in multiple studies including AF detection, coronary artery disease, stress and depression, and more. A patient wearable device assembly 300a-c may include: up to 96 hour rechargeable; 24 hour cache; IP25 water resistant; BLE network; size—90×20×7.9 mm; weight: 7.5 grams; 128 Hz ECG sensor; heart hate sensor 40 to 300 bpm; respiratory 5 to 35 brpm; 3-Axis ACC 5 Hz; FDA/NMPA-ECG; heart rate; and CE-ECG, heart rate, and respiratory rate.

A patient wearable device assembly 300a-c may include a SpO₂ sensor that may provide continuous updates of a patient's oxygen saturation levels without the need for the patient to manually initiate a reading. With a secure design that wraps around the thumb of a patient, the sensor may be to stay on the patient even in ambulatory situations. Advantages of any given patient wearable device assembly 300a-c may include: live stream or recording; reusable/rechargeable; IoT enabled; secure thumb strap; data including oxygen saturation, pulse, up to 16 hours rechargeable; 10 hour cache; IP 22; BLE network; weight: 47.5 grams; SpO₂—70% to 100%; pulse: 30 to 250 bpm; and FDA/CE/NMPA.

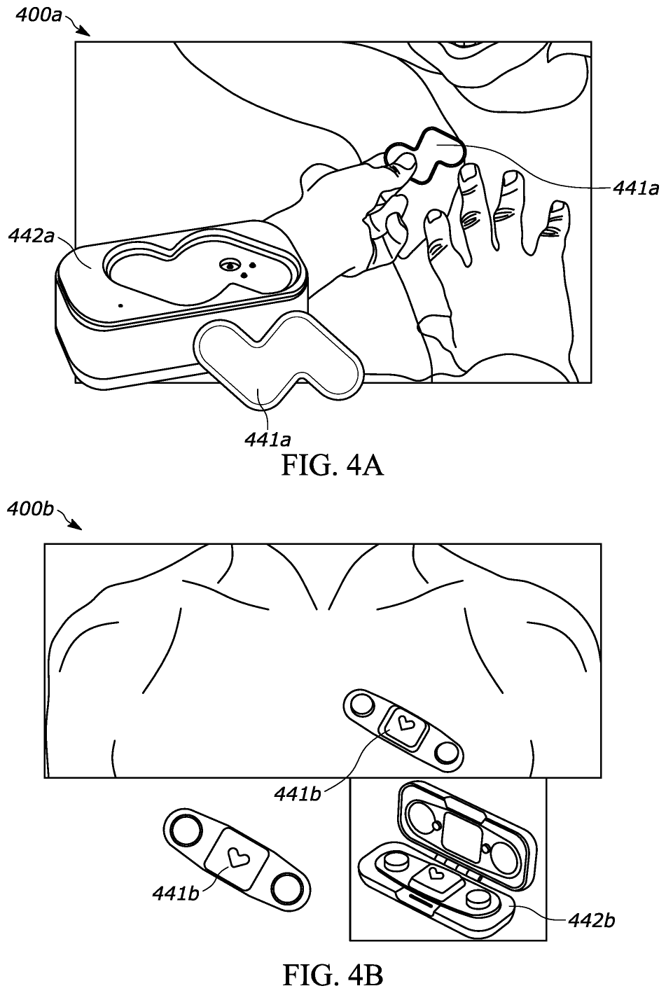

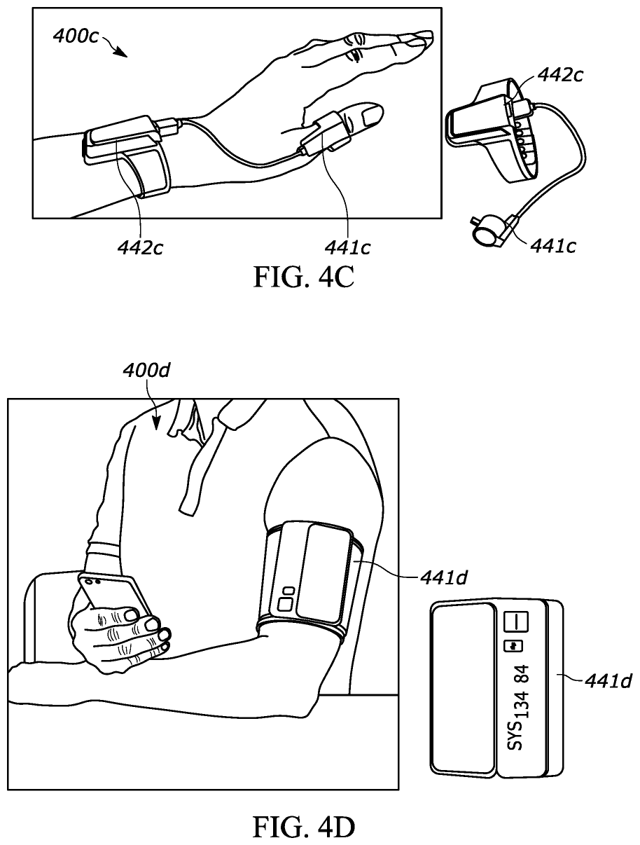

With reference to FIG. 4A, a sensor device assembly 400a may include an auxiliary temperature sensor 441a and a charging device 442a. The sensor device 441a may be similar to, for example, the sensor device 141 of FIG. 1 or 241a of FIG. 2A. Unlike typical body temperature patches which are not considered FDA clinical thermometers, the VivaLNK axillary temperature patch 400a may be a clinical thermometer that can provide a medically accurate (armpit) reading in ambulatory and remote patient monitoring settings. This sensor device assembly 400a may be used in clinical studies (e.g., complete out-patient treatment of patients having residual amounts of leukemia cells in their system, a likelihood of experiencing treatment-emergent adverse events (TEAE), such as, for example, cytokine release syndrome (CRS) and/or neurotoxicity (NT), a likelihood of experiencing other serious adverse events (SAEs) requiring hospitalization during the mandatory device monitoring period (MDMP), neurological drug development, chemotherapy remote patient monitoring, etc.). The sensor device assembly 400a may include: live data stream, data recording option, reusable, rechargeable, IoT enabled, water resistant, BLE booster, clinical grade axillary temperature, up to 21 day rechargeable, 20 hour cache option, IP25 water resistant, BLE network, size—61×41×5.5 mm, weight—7.2 grams, meets ASTM E1112 standard, range—93.2 F to 109.4 F, FDA/CE/NMPA, and pediatrics and adult use.

Turning to FIG. 4B, a sensor device assembly 400b may include a respiratory rate sensor 441b and a charging device 442b. With reference to FIG. 4C, a sensor device assembly 400c may include a heart rate sensor 441c and a wrist band 442c. The sensor device assembly 400b and/or the sensor device assembly 400c may be, for example, integrated into a patient wearable device 115 of FIG. 1 or 215a of FIG. 2A. Turning to FIG. 4D, a sensor device assembly 400d may include blood pressure sensor 441d. The sensor device 441d may be similar to, for example, the sensor device 140 of FIG. 1 or 240a of FIG. 2A.

Medications or factors related to hepatic injury/dysfunction and cholestasis or biliary obstruction may cause liver enzyme elevations above normal levels even in otherwise healthy individuals. An associated patient wearable device 115 and/or sensor device 140, 141 may generate a detailed serum chemistry, including, for example: liver enzymes such as alkaline phosphatase (AP), alanine aminotransferase (ALT), aspartate aminotransferase (AST), lactate dehydrogenase (LDH), and C-reactive protein (CRP), total bilirubin, gamma-glutamyl transferase (GGT), D-Dimer, a combination thereof, or any sub-combination thereof. Corresponding sensor data may, thereby, provide important information related to a respective patient's liver function in response to drug treatment, and may reveal drug induced hepatocellular, cholestatic or mixed liver injury. In any event, a content of the sensor data may be based on a respective medication and/or patient related bio-information. In general, an associated patient wearable device 115 and/or sensor device 140, 141 may be configured to sense body chemistry parameters that are pre-determined to be likely representative of a patient's reaction to a particular treatment and/or medication.

With reference to FIGS. 5A-H and J-L, various user interface displays of an out-patient treatment system 500a-h,j-l are depicted. As illustrated in FIG. 5A, a healthcare provider display device 500a may include a display of a patient 505a having a patient wearable device 515a, a sensor device 540a, an infusion pump 410a, and a patient device 550a. The display device 550b may include a patient vital signs 554b. The display device 580c may include healthcare provider status information 584c. The display device 580d may include a list of patients w/respective patient information 584d. The display device 580e may include a list of patients w/respective patient information 584e. The display device 580f may include patient information 584f. The display device 580g may include patient information 584g. The display device 550h may include a patient on a video call with a healthcare provider 554h. The display device 580j may include a healthcare provider on a video call with a patient 584j. The display device 550k may include a patient-initiation call button 554k. The display device 550l may include an incoming video call to a patient from a respective care team 5541.

### Example Study

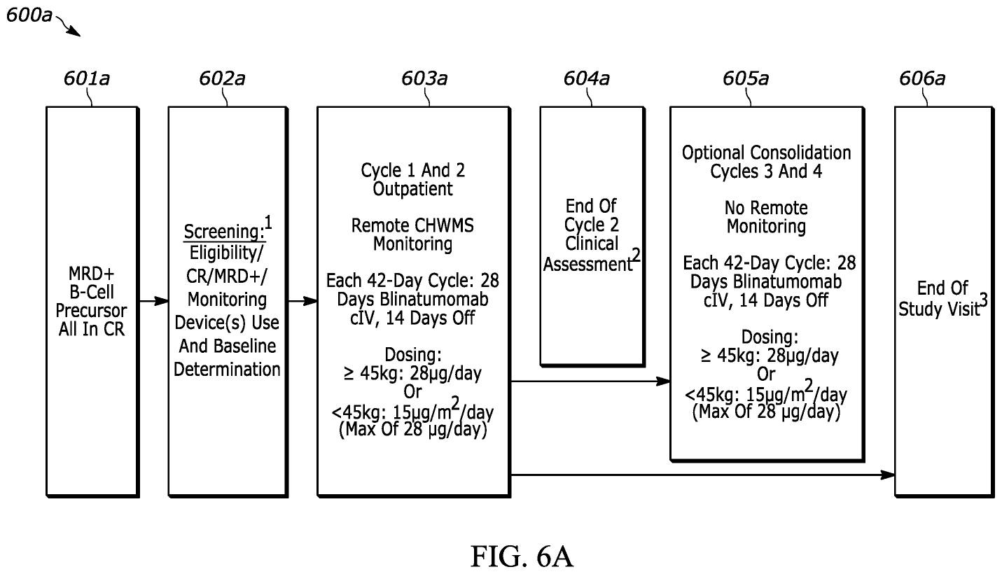

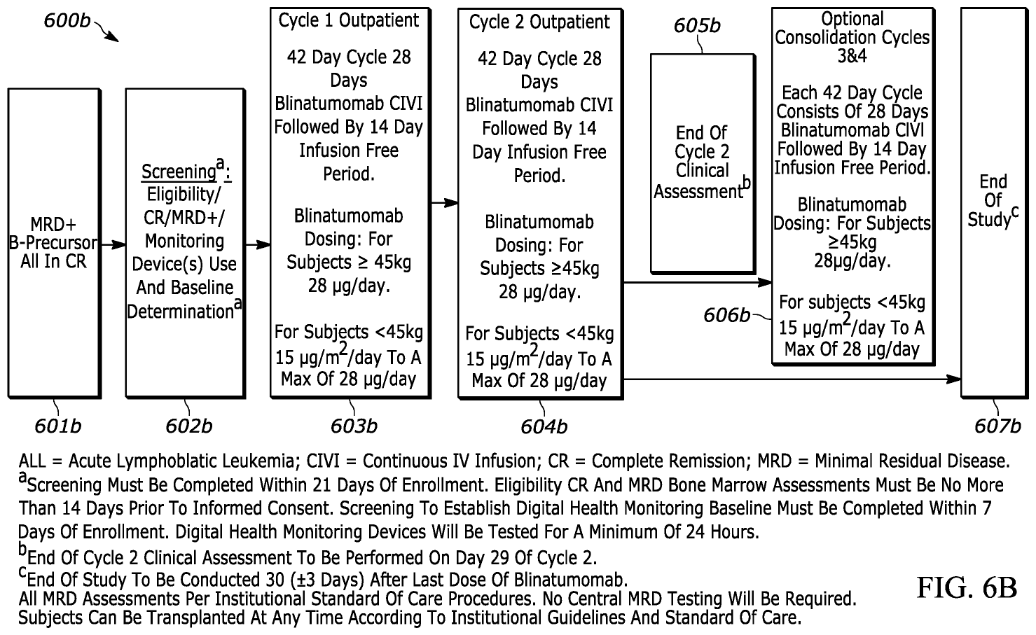

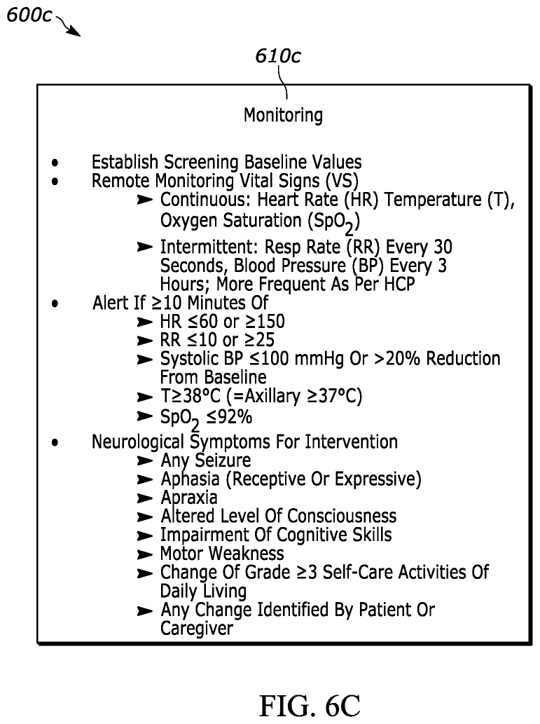

With reference to FIGS. 6A-C, the apparatuses, systems, and methods of the present disclosure were applied to a phase 4, multi-center open-label feasibility study (i.e., EXAMPLE STUDY) to evaluate out-patient blinatumomab administration in adult subjects with minimal residual disease (MRD) of B-precursor acute lymphoblastic leukemia (ALL) in complete hematologic remission.

The example study included a first sub-study 600a having: a patient examination stage 601a, a patient screening stage 602a, a first medication administration stage 603a, a medication administration stage end 604a, a second medication administration stage 605a, and an end of first sub-study 606a. The example study included a second sub-study 600b having: a patient examination stage 601b, a patient screening stage 602b, a first medication administration stage 603b, a second medication administration stage 604b, a medication administration stage end 605b, a third medication administration stage 606b, and an end of second sub-study 607b. The example study included patient 105 monitoring 600c having a plurality of patient monitoring requirements 610c.

The phase 4 indication included adult subjects with MRD of B-precursor ALL. Rationale for this example study was to determine the safety and feasibility of complete outpatient blinatumomab administration for subjects with MRD of B-precursor ALL. Blinatumomab administration had been demonstrated to be efficacious in the respective population for converting subjects to MRD negative status (MRD<0.1%). However, the mechanism of action (MOA) of T cell activation and cytokine production can result in potentially severe toxicity, specifically cytokine release syndrome (CRS) and/or neurotoxicity (NT). The incidence of these potentially severe side effects had been shown to be low. In the BLAST study, 3% and 13% of subjects had severe CRS and NT respectively (e.g., Gökbuget et al, 2018).

The objective of this example study was to determine the safety and feasibility of complete outpatient blinatumomab administration for subjects with MRD of B-precursor ALL. The example study used mobile electronic devices (e.g., a tablet, a smartphone, a personal electronic device, etc.) to electronically communicate with an associated healthcare provider (HCP), and a Wi-Fi enabled platform for real-time constant transfer of data and communication between subject and HCP to detect clinically important changes. The data provided to the HCP may enable the HCP to identify subjects at risk of developing grade 3 or 4 CRS, NT, or other serious adverse events (SAEs) requiring hospitalization during the mandatory device monitoring period (MDMP). In circumstances where the subject experiences a SAE, the HCP can then direct such subjects to the appropriate medical facility for hospitalization if needed.

TABLE 1
Example Study Objectives and End Points
Objective(s)/Endpoint(s)

| | |
|---|---|
| Objectives | Endpoints |

Primary

| | |
|---|---|
| To determine the safety of outpatient | Incidence of grade 3 and/or |
| blinatumomab administration, during | 4 CRS, NT or any adverse |
| the MDMP (defined as the first 3 days | event (AE) requiring |
| of cycle 1 and first 2 days of cycle 2 of | hospitalization (SAE) during |
| outpatient blinatumomab infusion) | MDMP. |

Secondary

| | |
|---|---|
| To determine the time from first | Time (in minutes) from first |
| detection of grade 3 or 4 vital sign, or | onset of fever, hypotension, |
| significant clinical change to | hypoxia, other grade 3 or |
| therapeutic intervention (any | 4 vital sign including seizure or |
| measurable action taken by or | neurological change (grade |
| performed on the subject due to onset | 3-limiting self-care activities of |
| of the clinical parameters described | daily living [ADL]) to |
| above) during the MDMP | therapeutic intervention^{a} |
| Evaluate the safety and tolerability of | Overall incidence and severity |
| blinatumomab administered | of treatment-emergent |
| exclusively as an outpatient | adverse events (TEAE) and |
| | adverse events of interest in |
| | particular CRS and NT. |
| Evaluate the impact of complete | European Organisation for |
| outpatient blinatumomab treatment on | Research and Treatment of |
| patient-reported outcome (PRO), | Cancer (EORTC) validated |
| global health status (GHS), and | electronic version of |
| quality of life (QoL). | QLQ-C30. |
| Estimate healthcare resource | TEAEs that resulted in |
| utilization associated with | hospitalizations |
| treatment-related AEs | TEAEs that resulted in |
| | surgeries. |
| | TEAEs that resulted in the use |
| | of concomitant medications |
| | TEAEs that resulted in the use |
| | of device/procedure |
| | intervention. |

ADL = activities of daily living; AE = adverse event; CG = caregiver; CRS = cytokine release syndrome; EMS = Emergency Medical Service; EORTC = European Organisation for Research and Treatment of Cancer; GHS = global health status; HCP = Health Care Provider, IV = intravenous; MDMP = mandatory device monitoring period; NT = neurotoxicity; PO = by mouth; PRO = patient-reported outcomes; QLQ-30 = Quality of Life of Cancer Patients; QoL = quality of life; SAE = serious adverse event; TEAE = treatment-emergent adverse event.
^{a}Therapeutic intervention is any measurable action taken by the subject or performed on the subject as a result of the onset of the clinical parameters described. Such actions may include: Advice from HCP to immediately call EMS or dial 911, discontinuation of the blinatumomab infusion, subject taking an oral medication, subiect receiving a medication or intervention by any emergency or hospital medical services, intervention such as administering any medication (IV or PO or PR) or IV fluids or oxygen etc Time to therapeutic intervention (TTI) is the measured time between the delivery of the device alert or unscheduled contact with HCP by subject/CG to report a change to the time of initiation of the therapeutic intervention as defined.

The example study determined the safety and feasibility of complete outpatient blinatumomab administration. Blinatumomab is a novel bispecific T-cell engaging (BiTE®) single-chain bispecific binding molecule construct that links CD3+T lymphocytes with CD19+ B cells. This treatment may result in a significant degree of T cell-mediated immune activation in subjects, which has correlated with efficacy but also with notable toxicity. The result of the example study included a heightened T-cell activation and release of pro-inflammatory cytokines and the clinical manifestation of CRS. Along with CRS, another potentially severe toxicity observed is NT. It was anticipated that some subjects may show signs of encephalopathy with varying degrees of severity and may experience delirium, aphasia, lethargy, difficulty concentrating, agitation, tremor, seizures, and, rarely, cerebral edema.

Minimal residual disease (MRD) is defined as the presence of leukemic cells not detectable by microscopy (<5%) with a sensitivity of 0.1%, measured either by polymerase chain reaction (PCR) or flow cytometry. After achieving hematologic remission, the presence of MRD portends a poor prognosis for patients. In an associated Amgen trial (i.e., MT103-203 (BLAST)), 78% of subjects with MRD of B-precursor ALL achieved a complete MRD response to blinatumomab therapy. In this trial, 4 subjects (3%) had CRS (Common Terminology Criteria for Adverse Events [CTCAE] v4 grade 1, n=2; grade 3, n=2), all during cycle 1. Twelve (10%) and 3 subjects (3%) had grade 3 and 4 NT, respectively. Moreover, subjects with relapsed/refractory B-precursor ALL treated with blinatumomab in the Amgen trial 00103311 (TOWER) similarly had a low incidence of CRS and NT. In the 00103311 study the incidence of grade 3 CRS and NT were 4.9% and 9.4%, respectively with 1% and 4.9% of subjects having their treatment discontinued due to CRS and NT, respectively (i.e., Kantarjian et al, 2017).

At the time of the study, the current recommendation for blinatumomab treatment for subjects with MRD of B-precursor ALL was continuous intravenous infusion (CiVI) for 28 days, with the first 3 days in cycle 1 and the first 2 days in cycle 2 administered in the inpatient setting. This recommendation for hospitalization is primarily for safety concerns of CRS, NT, or other severe AEs.

Implementing the apparatuses, systems, and methods of the present disclosure, this example study determined the safety and feasibility of complete outpatient blinatumomab administration for subjects with MRD of B-precursor ALL. A patient (e.g., patient 105 of FIG. 1) wore a patient wearable device (e.g., patient wearable device 115 of FIG. 1, 215a of FIG. 2A, 215d of FIG. 2D, 215m of FIG. 2M, 215n of FIG. 2N, or 315a-c of FIGS. 3A-C, respectively, a patient wearable device 215m,n as available from Current Health device (https://currenthealth.com/), a patient wearable device 300a,b of FIGS. 3A and 3B, respectively, a patient wearable device 300c of FIG. 3C, as available from Bio Beat (https://www.bio-beat.com/), etc.). The patient wearable device was worn on an upper arm 115 as illustrated in FIG. 1.

Additionally, each patient 105 wore at least one sensor device (e.g., a patient wearable sensor device 140 of FIG. 1, 240a of FIG. 2A, 240b of FIG. 2B, 240m, 241m of FIG. 2M, 240n, 241n of FIG. 2N, or 440a-d of FIGS. 4A-D, respectively). Subjects of this example study, wore an auxiliary temperature patch (e.g., similar to a Fever Scout auxiliary temperature patch 441a, as available from VivaLNK) and a blood pressure cuff (e.g., similar to an Evolv sensor device 441d, as available from Omron).

Each patient was provided, a Wifi hub (e.g., network 250a of FIG. 2A, a hub 295m of FIG. 2M or 295n of FIG. 2N as available from) is configured to securely communicate patient related data (e.g., sensor data, warning data, call initiation data, audio/video data, etc.).

Additionally, each patient was provided a patient device (e.g., a tablet device 150 of FIG. 1, 250a of FIG. 2A, 250f of FIG. 2F, 250m of FIG. 2M, 250n of FIG. 2N, 450a of FIG. 4A, 450h of FIG. 4H, 450j of FIG. 4J, 450k of FIG. 4K, etc.) w/software module (e.g., module 253a of FIG. 2A, 253f-261f of FIG. 2F, etc.) (e.g., ePro software module, as available from Ennov, Paris, France—CLinical suite-Ennov ePRO-Electronic Patient Reported Outcomes Software, etc.). The software module 253a may, when executed by a processor (e.g., processor 251a of FIG. 1A), cause the processor 251a to implement regulated content, data and process management (e.g., communication, storage, access to patient specific information—electronic health records, sensor data, etc.). For this example study, the module 253a was IS09001:2015 certified for all associated apparatuses, systems, computer-readable medium, and methods. The module 253a included enterprise document management (EDM), regulatory information management (RIM), pharmacovigilance, and electronic trial master files (eTMF).

Each healthcare provided was provided a mobile phone (e.g., smartphone 450g,j,k of FIGS. 4G, J, and K, respectively). Additionally, each patient was provided a module (e.g., module 283a of FIG. 2A (e.g., a platform as available from Current Health device (https://currenthealth.com/), a platform available from Bio Beat (https://www.bio-beat.com/), or any other suitable remote and/or wearable monitor based platform) is used to monitor subjects' (e.g., patient 105 of FIG. 1) vital signs while the subjects 105 were at home (e.g., patient's home 100 of FIG. 1).

This example study included the following: 1) remote monitoring to measure vital signs (e.g., sensor data) and mobile electronic devices (e.g., a patient device 250a with module 253a) to electronically communicate the sensor data with the HCP (e.g., 280a of FIG. 2A, 484h of FIG. 4H, etc.); 2) use of a Wi-Fi enabled platform for real-time continuous transfer of patient related data, and audio/video communication between subject 105 and HCP 484h, to identify subjects that were at risk of developing grade 3 or 4 CRS, NT or other SAEs requiring hospitalization during the MDMP. The subject group of this example study may require immediate escalation of care and/or hospitalization once the digital monitoring system (e.g., system 200a of FIG. 1A) identifies changes.

During screening within the example study, subjects and caregivers were trained on the patient wearable device 115, and were assessed for compliance. Subjects receive 2 cycles of blinatumomab completely in the outpatient setting in accordance with the monitoring and intervention guidelines 510c of FIG. 5C. The end of study visit occurs 30 days (3 days) after the last dose of blinatumomab FIGS. 6A and B.

After the end of cycle 2 clinical assessment visit, some subjects may continue to receive 2 additional (optional) cycles of blinatumomab. There is no health management system (HMS) 270a 270a outpatient monitoring during the optional cycles 3 and 4. For the purpose of this study, the MDMP is defined as the first 3 days of cycle 1 (72 hours) and the first 2 days of cycle 2 (48 hours) of blinatumomab infusion for subjects with MRD of B precursor ALL.

The end of study visit occurs 30 days (3 days) after the last dose of blinatumomab is given. The end of study visit occurs after cycle 3 or 4 for subjects who chose these optional cycles. During the MDMP, the health management system (HMS) 270a 270a measures vital signs. These vital signs include heart rate (HR), axillary temperature, and oxygen saturation. The health management system (HMS) 270a measures respiratory rate (RR) intermittently (sampling every 30 seconds). The subjects take intermittent blood pressure (BP) measurements (using a subject-usable BP device) every 3 hours during the MDMP. The schedule for BP measurements could have been extended by HCP (up to but not exceeding every 6 hours) after the first 24 hours of MDMP. The BP device 140 be provided and directly transmits the BP reading via the monitoring platform to the HCP device 270a.

Threshold vital sign values were established, and an immediate alert was generated, and transmitted to the HCP device 280a once the preset threshold values were surpassed and sustained for at least 10 minutes. In addition, subject and caregiver were trained on the usual side effects of blinatumomab infusion (fever, erythematous skin rash, chills, confusion, headache, tremor, myalgia, lethargy, somnolence, seizure), and have direct (phone and video) contact with HCP or emergency services if any expected or unexpected side effects occur. The caregiver is expected to be a spouse or close relative such as a child but included any adult (18 years) willing and able to participate in the subject's care. The caregiver remains in the home with the subject for the entire MDMP. The caregiver is trained to use and have access to the patient device (e.g., tablet 150 of FIG. 1) for communication with the HCP, if necessary.

The HCP and the staff were trained on the infusion 110, usual side effects and response algorithm. The HCP (or designee) is a physician with experience in the treatment of patients with ALL and the use of blinatumomab. The HCP (or designee) carries the smart phone 480h device at all times during the MDMP. The smart phone 480h includes a cellular connection to the subject's tablet 150 and the health management system (HMS) 270a 170 and receives vital signs (i.e., sensor data) refreshed every 30 seconds for the entire MDMP. Blood pressure is performed manually by the subject (or caregiver) every 3 hours and the result electronically delivered to the HCP smart phone 480h. In addition to the continuous feed of vital signs, an audible alarm sounds every time the vital signs exceed the preset threshold and is consistent for at least 10 minutes. In addition, an audible alarm sounds if there is no data transfer for 15 minutes.

It is not intended that the health management system (HMS) 270a 170 allows the HCP to identify patients experiencing neurotoxicity. The health management system (HMS) 270a is intended to provide remote monitoring of vital signs. Knowing a subject's vital signs provide the HCP an overall picture of the subject's disposition. In addition to vital signs monitoring, the health management system (HMS) 270a includes the functionality for video calling between the physician and the subject. This allows the HCP to visually assess the patient for early symptoms of mild neurotoxicity such as kinetic tremor (assessed by finger-nose test), ataxia, appearance of disorientation to time or place, impaired attention or short-term memory with preserved alertness, impaired naming, paraphasic errors, or verbal perseveration. The HCP also tests the patient's ability to name objects, follow simple commands, and communicate their needs. The HCP evaluates expressive aphasia by evaluating the subjects' ability to communicate spontaneously (for example HCP may instruct subject to “look directly on the video screen and tell me how you are feeling today”) or in naming a common household object provided by caregiver. HCP evaluates apraxia by the subject's ability to write a standard sentence (for example “Today is Tuesday Jan. 1, 2001”). HCP tests receptive aphasia by the subject's ability to follow a simple command (for example “Lift your right hand and touch your nose”).

In addition, the caregiver is present with the patient for the entire MDMP and is able to communicate and participate with the HCP during the video call assessment. The HCP uses the video call assessment to determine if the patient's symptoms (as defined for neurotoxicity) necessitate immediate transfer to hospital. The HCP schedules video calls with the patients a minimum of every 12 hours (e.g., 8 am and 8 pm) daily during the MDMP. The frequency was increased when there were any concerns by the HCP.

The monitoring system also provides the functionality for the patient or caregiver to activate an “I don't feel well” button 320b on the device tablet 170 in the event of the subject experiencing any neurological symptoms. Activating the button 320b immediately generates an alarm and notifies the HCP and allows for an immediate video call assessment to be initiated. HCP advises transfer to an inpatient facility if any of the neurological symptoms were greater than mild in severity. In addition, as part of screening, patients and caregivers were trained to activate the “I don't feel well” button 320b for any moderate symptoms of speech disorders, disturbances in consciousness, deterioration of hand writing, confusion or disorientation, hypotension, hypoxia, transaminitis, hypertension, vomiting, diarrhea, or other symptoms that may be associated with a CRS.

Subjects were trained to resend the “I don't feel well” signal if no response from the HCP is received in 5 minutes. If there is still no response after another 5 minutes, 10 minutes total from the initial alarm, then the subject was to call emergency medical service (EMS) or dial 911 immediately. Likewise, when the HCP responded to any alarm generated from the subject and the subject or caregiver did not answer within 10 minutes, then the HCP made 1 attempt to call the subject's personal phone. If the subject or caregiver was still not reachable, the HCP had EMS dispatched to the subject's location immediately. The HCP phone is configured so that a loud audible alarm is made and repeated every 5 minutes until the HCP responds or if no data is transferred for 15 minutes. The HCP then used the compendium of vital sign's absolute value and/or deviation from baseline (provided by the health management system (HMS) 270a) and the subject's clinical status (provided by direct subject/caregiver telephone and video contact), to make decisions on urgency of response, appropriate intervention and subject disposition.

During MDMP any device malfunction (e.g., a patient wearable device malfunction, a sensor device malfunction, an axillary temperature patch malfunction, a blood pressure sensor malfunction, a communication network malfunction, an in-home hub malfunction, etc.) that is detected within 15 minutes generated an alarm to the HCP. The HCP contacts the patient immediately via the video calling functionality to assess the patient's safety, clinical status, and any specific circumstances associated with the device malfunction (e.g., device inadvertently dislodged, low battery charge, intermittent loss of signal transmission etc.) If the HCP has any concern of patient safety, HCP advises the patient to immediately go to the hospital.

Patients were provided with a full set of replacement devices to be used in case of a malfunction. In addition, a manual oral thermometer is provided to the patients, as needed or directed by the HCP. In the case of a device malfunction and the HCP decides that the patient is safe via the video conference, the HCP directs the patient to switch to the replacement device(s) immediately. In addition, HCP may contact a health monitoring platform 270a (e.g., Current Health's 24/7 hotline, etc.) for device support and troubleshooting for assistance. It is recommended that the HCP remain in contact with the subject until device malfunction is resolved. If these interventions by HCP fail to resolve the device malfunction, then HCP advises the patient to proceed immediately to the hospital. Only United States (US) Food and Drug Administration (FDA) cleared vital sign monitoring devices and platform 270m used. During the MDMP, subjects were required to live no more than 1-hour transportation by car from an advanced medical care facility. An advanced medical care facility included staff trained to manage acutely ill patients such as those who may have developed CRS. Subjects were allowed to stay at a hotel or other outpatient hospital housing facilities to fulfil the 1-hour transportation requirement. Subjects were trained to call emergency medical services for any concerns or for any delay in communication with the HCP.

Subjects were provided with duplicate devices (e.g., BP monitor, axillary temperature patch, Current Health wearable device) of the health management system (HMS) 270a 270m in case of unexpected device malfunction (see above). In addition, subjects were provided with a dedicated in-house Wi-Fi device (home hub 295m,n) for the sole purpose of uninterrupted transmission of vital signs and for communication including video contact with the HCP. Subjects, caregivers, and HCP have access to a 24/7 helpline for any technical issues relating to the home digital monitoring devices, platform or communication.

Alternative monitoring devices may be used, such as a Biobeat monitoring device (https://www.bio-beat.com/), which may be used to measure pulse pressure, continuous blood pressure, blood saturation, pulse rate, respiratory rate, systematic vascular resistance, heart rate variability, arterial pressure, skin temperature, heart stroke volume, cardiac output, and cardiac index. The Biobeat device may be able to communicate with smart devices or otherwise provide continuous or semi-continuous wireless monitoring features. The device may be self-adhesive and may have disposable chest patches and/or a cuff accessory.

With further reference to FIG. 6B, a study schema 600b includes example study endpoints. There was no outpatient digital monitoring during the optional cycles 3 and 4. Participants in this clinical investigation shall be referred to as “subjects.” Approximately 45 subjects participated in the example study. Adult subjects have MRD of B-precursor ALL in complete hematologic remission (<5% bone marrow blast cells). Amgen Investigational Product Dosage and Administration: Blinatumomab is administered as a CiVI. A single cycle of blinatumomab treatment is 6 weeks in duration, which includes 4 weeks of blinatumomab CiVI followed by a 2-week treatment-free interval. The treatment-free interval was prolonged by up to 7 days, if deemed necessary by an investigator. For subjects 45 kg, blinatumomab was administered at a dose of 28 μg/day for all 4 weeks of continuous treatment. Subjects <45 kg were administered blinatumomab 15 μg/m2/day (max 28 μg/day).

Non-investigational Product Dosage and Administration included: Dexamethasone-Premedication with dexamethasone (or prednisone): Dexamethasone 16 mg (or equivalent prednisone 100 mg) intravenously (IV): up to 6 hours before start of treatment in each treatment cycle. Subjects 45 kg had 2 additional 8 mg doses (subjects <45 kg receive 2 doses of 5 mg/m2 rounded to the nearest mg) of oral dexamethasone to use as needed for treatment of CRS symptoms only if instructed by HCP. It was recommended that hospitalization was strongly considered for subjects who required interruption of blinatumomab for >4 hours.

Written informed consent was obtained from all subjects before any study-specific screening procedures were performed. Study-specific procedures occurred according to the Schedule of Assessments FIGS. 6A and 6B. The planned enrollment for the example study was 45 patients. The sample size was based on feasibility rather than on statistical considerations. Given a true probability of a primary endpoint event of 38%, the expected 95% upper confidence limit with 45 subjects would be 53.5%. The 38% subject incidence rate was based the SAE rate in first 3 days of cycle 1 and first 2 days of cycle 2 from the previous MRD study (BLAST). Based on these assumptions, we expect to observe approximately 17 subjects who experienced the primary endpoint which provided an initial indication of the timeliness of the therapeutic intervention when outpatient blinatumomab in administered in conjunction with remote digital monitoring. Safety data was reviewed on an ongoing basis. A data review team (DRT), internal to Amgen but external to the study team, assessed safety after every 5 subjects had the chance to complete the mandatory device monitoring period. The primary analysis occurred when all subjects ended the example study. The primary analysis was also the final analysis.

Descriptive statistics for demographic and safety were summarized as appropriate. For categorical variables, the number and percentage of subjects in each category were summarized. Continuous variables were summarized by n, mean, standard deviation, median, Q1 (25th percentile), Q3 (75th percentile), minimum, and maximum values.

The example study determined the safety and feasibility of complete outpatient blinatumomab administration for subjects with minimal residual disease (MRD) of B-precursor acute lymphoblastic leukemia (ALL). Blinatumomab administration has been demonstrated to be efficacious in this population for converting subjects to MRD negative status (MRD <0.1%). However, the mechanism of action (MOA) of T cell activation and cytokine production can result in potentially severe toxicity, specifically cytokine release syndrome (CRS) and/or neurotoxicity (NT). The incidence of these potentially severe side effects has been shown to be low. In the BLAST study, 3% and 13% of subjects had severe CRS and NT respectively (i.e., Gökbuget et al, 2018).

The objective of the example study was to determine the safety and feasibility of complete outpatient blinatumomab administration for subjects with MRD of B-precursor ALL. The study used mobile electronic devices (tablet 170) to electronically communicate with the HCP, and a Wi-Fi enabled platform 200a for the real-time constant transfer of data and communication between subject and HCP 250a to detect clinically important changes. The data provided to the HCP enabled the HCP to identify subjects at risk of developing grade 3 or 4 CRS, NT, or other serious adverse events (SAEs) requiring hospitalization during the mandatory device monitoring period (MDMP). The HCP then directed such subjects to the appropriate medical facility for hospitalization when needed. The population for this study (MRD of B-precursor ALL) had a low incidence of severe CRS and NT and a reasonable target population to evaluate the feasibility of complete outpatient blinatumomab administration.

The example study used a Current Health (CH) system 270m and platform for outpatient monitoring. Subjects who were in a high-acuity environment, such as an operating room (OR) or intensive care unit (ICU), or those who were acutely ill and that developed immediate and life-threatening arrhythmias were excluded from the CH platform and not enrolled into the example study. As illustrated in FIGS. 6A and 6B, the primary endpoint of the example study was an incidence of grade 3 or 4 CRS, NT or any adverse event (AE) requiring hospitalization during the MDMP. The CH devices detects abnormal vital signs or changes in baseline vital signs to allow the HCP to determine if subjects developed CRS. In addition, the CH device contain functionality for video calling between the HCP and the patient to allow the HCP to visually assess the patient for early symptoms of mild neurotoxicity. The vital signs were all monitored by the health management system (HMS) 270a with performance assessed and cleared by the United States (US) Food and Drug Administration (FDA) as non-inferior to both Draeger and Philips vital signs monitoring equipment. Thus, the CH devices were appropriate and applicable for the monitoring of abnormal vital signs in the home environment.

B-precursor ALL is a malignant disease of lymphatic progenitor cells in the bone marrow or sites of lymphatic system. Immature lymphoblasts proliferate in the bone marrow and may infiltrate other organs. As a consequence, the normal hematopoiesis in the bone marrow is suppressed. Acute lymphoblastic leukemia is a rare malignant disease with an overall incidence of 1.1/100,000 per year. Acute lymphoblastic leukemia has a bimodal distribution with an early peak at 4 to 5 years of age (incidence of 4.5/100,000 per year) followed by a second gradual increase at 50 years (incidence of 2/100,000 per year). It represents 80% of acute childhood leukemia and 20% of acute leukemia cases in adults (i.e., Pui and Evans 1998; Jabbour et al, 2005; Larson 2005; Howlader et al, 2012).

Seventy-5 percent of adult subjects with ALL is of B-cell lineage and approximately 25% is derived from T-cell lineage. The majority of subjects with B-cell lineage ALL have an immature immunophenotype and were classified as B-precursor ALL. CD19 is expressed in all subtypes of B-ALL (Bassan et al, 2004). The Philadelphia chromosome (Ph) represents the most frequent cytogenetic aberration in adult ALL and is found in 20-30% of subjects with B-precursor ALL. Adult ALL can be stratified into risk groups that were the basis for risk adapted treatment strategies. A selection of prognostic factors in B-precursor ALL: Prognostic Factors for Risk Stratification of Adult B-precursor ALL.

TABLE 2
Prognostic Factors for Risk Stratification of Adult B-precursor
ALL (according to Gökbuget and Hoelzer 2009)

| Parameter | Favorable | Adverse |
|---|---|---|
| WBC at diagnosis | <30,000/μl | >30,000/μl |
| Immunophenotype | | Pro-B-ALL |
| Cytogenetics/Molecular | | t (9; 22)/bcr-abl |
| genetics | | t (4; 11)/ALL1-AF4 |
| Time to CR | Early | Late (>3-4 weeks) |
| MRD after induction and | Negative (<10⁻⁴) | Positive (>10⁻⁴) |
| consolidation | | |
| Age | <25 years, <35 years | >35 years, >55 years, |
| | | >70 years |

ALL = acute lymphoblastic leukemia; CR = complete remission; MRD = minimal residual disease; WBC = white blood cell count

A white blood cell count (WBC) of <30,000/1 at diagnosis and younger age were favorable factors in adult ALL. Additionally, a short interval until achievement of a complete remission (CR) and a complete molecular remission following induction (MRD negative; <1 leukemic cell in 104 bone marrow cells is detectable) were favorable factors. Outcome of therapy in adult patients with ALL has improved substantially over the last 10 years with CR rates of 85 to 90% and overall survival (OS) rates of 40 to 50% (Gökbuget and Hoelzer, 2010). Pediatric protocols have been the guideline to develop treatment regimens for adult ALL. These regimens consist of 3 phases: induction, consolidation, and maintenance therapy. Generally, the therapeutic “state of the art” modalities for adult leukemia can be summarized (Gökbuget and Hoelzer, 2011). 4- to 5 drug induction regimens using vincristine and prednisone, adding anthracyclines, cyclophosphamide, asparaginase (or a combination of these agents) to achieve CR. Intensified consolidation therapy based on cyclical administration of cytarabine, anthracyclines, methotrexate, asparaginase and repeated induction to reduce the level of MRD.

Protracted maintenance therapy (about 2 years) using methotrexate combined with mercaptopurine; allogeneic stem cell transplantation in first CR in high-risk patients: Allogeneic hematopoietic stem cell transplantation (HSCT) is the most intensive option of consolidation in first CR of high-risk patients. Progress in therapy and optimized risk stratification, which enabled patient-tailored treatment, were important factors contributing to improvement of treatment outcome. In spite of the success in upfront treatment, the outcome in most adults with recurring disease irrespective of their prior treatment is dismal, as they cannot be rescued using currently available therapy (Fielding et al, 2007). Thus, prevention of relapse is the major treatment goal in patients resistant or intolerant to chemotherapy.

The platform illustrated in FIGS. 2M and 2N, and as implemented in the example study, is an FDA-cleared platform for wireless and wearable health monitoring of patients at home and hospital. The CH product was launched on the US market in December 2018 after attainment of FDA clearance. With respect to the example study, Current Health's FDA cleared indications for use were as follows: the health management system (HMS) 270a was intended for reusable bedside, mobile and central multi-parameter, physiologic patient monitoring of adult patients in professional healthcare facilities, such as hospitals or skilled nursing facilities, or the patient's own home. It was intended for monitoring of patients by trained HCPs. The health management system (HMS) 270a was intended to provide visual and audible physiologic multi-parameter alarms. The health management system (HMS) 270a is intended for continuous monitoring of the following parameters in adults: heart rate (HR), axillary temperature, oxygen saturation, skin temperature (not evaluated for this study), movement (not evaluated for this study), and spirometry (not evaluated for this study)

In the context of the example study, the health management system (HMS) 270a was intended for intermittent or spot-check monitoring, in adults, of: respiratory rate (RR) (every 30 secs); non-invasive blood pressure (BP); lung function & spirometry (not collected for this study); and weight (not collected for this study). The health management system (HMS) 270a generated intermittent respiration rates by sampling sensor data every 30 seconds. This meant that every 30 seconds new respiration rates were available for use in the alarm system and for presentation on an associated dashboard. This functionality was assessed using physician-underscored end tidal CO₂ in CH 510(k) K182453. The health management system (HMS) 270a functioned as a hub with seamless, wireless integrations. Data flows directly through the CH wearable via integrated third-party devices such as: the axillary temperature monitor and the BP monitor. For the example study, the following was monitored and collected: heart rate (HR), axillary temperature respiratory rate (RR), oxygen saturation, and blood pressure (BP). The health management system (HMS) 270a was not intended for use in high-acuity environments, such as ICUs or ORs, or for use on acutely ill cardiac patients with the potential to develop life threatening arrhythmias eg, very fast atrial fibrillation and is not intended for peripheral capillary oxygen saturation (SpO₂) monitoring in conditions of high motion or low perfusion. These excluded patients and conditions were not be eligible for this example study.

Additional detail related to the health management system (HMS) 270a and integrated platform is described herein. Blinatumomab is a CD19-directed bispecific single-chain bispecific binding molecule construct designed to link B cells and T cells resulting in T-cell activation and a cytotoxic T-cell response against CD19 expressing cells. As stated previously blinatumomab is approved for the treatment of MRD of B-precursor ALL and has shown efficacy in the recently completed BLAST trial (Gökbuget et al, 2018). The approval for blinatumomab recommends that the initiation of cycle 1 (first 3 days) and of cycle 2 (first 2 days) be given while patients are admitted to hospital. In this investigational study we are testing the hypothesis that blinatumomab can be given as an outpatient for the entire 28 days if subjects are monitored during the initiation days. The blinatumomab infusion, doses, and indication for use were consistent with an associated FDA approved label.

Results from previously conducted pivotal Study M103-203 and supportive Study M103-202 showed similar high (up to 80%) MRD response rates after blinatumomab treatment in subjects with ALL in complete hematologic remission (<5% bone marrow blasts) who were MRD positive at the start of blinatumomab treatment baseline. Minimal residual disease response is a strong prognostic factor for relapse after achieving CR regardless of treatment choice or risk classification system (Lussana et al, 2016; van Dongen et al, 2015; Gökbuget et al, 2012; Gökbuget and Hoelzer, 2011; Bassan et al, 2009; Brüggemann et al, 2006). At the time of relapse, the strongest prognostic factors for OS are duration of initial remission and age (i.e., Oriol et al, 2010; Fielding et al, 2007; Thomas et al, 1999).

A published meta-analysis of 16 studies in adults with ALL solidified the association between MRD-negative status and improved clinical outcomes (event-free survival [EFS] and OS) that has up to this point been based largely on reports of individual studies with variable sample sizes. Of particular interest, EFS and OS for subjects who are MRD negative (MRD <0.1%) were shown to be remarkably consistent across a variety of subgroups (eg, MRD detection by flow cytometry vs polymerase chain reaction (PCR), MRD assessment after induction vs after consolidation, definition of MRD positivity, Ph status, and B-cell or T-cell phenotype) (i.e., Berry et al, 2017).

Key safety factors, that had been identified in clinical trials, were (and are) NT, CRS, and medication errors. Most AEs occurred within the first few weeks of the first cycle and were mitigated by appropriate measures such as temporary interruption without negatively affecting therapeutic benefit. Based on the short half-life of blinatumomab, in the presence of an AE, blinatumomab can be rapidly discontinued and cleared, which may enhance the ability to manage the AE effectively. In addition, the rate of severe AEs, in particular, CRS, are low enough to warrant the evaluation of complete outpatient administration of blinatumomab. This is particularly so in the MRD positive population, who by definition have low tumor burden. Moreover, the CH system is FDA cleared to accurately assess vitals and transmit vital signs efficiently to an HCP. In addition to benefit of MRD treatment, full outpatient blinatumomab is anticipated to be more convenient for patients.

The above benefit risk assessment supports the conduct of this clinical trial (i.e., the example study). Reference was made to an Investigator's Brochure for further data on blinatumomab. Training material was provided by CH with further information on the digital devices used in the study. The example study determined the safety and feasibility of complete outpatient blinatumomab administration for subjects with MRD of B-precursor ALL using health management system (HMS) 270a to measure vital signs and transmit the vital signs to the HCP, mobile electronic devices (e.g., a tablet 150 of FIG. 1) to electronically communicate with the HCP, and a Wi-Fi enabled platform (e.g., platform 500m,n of FIGS. 5M and 5N) for the real-time continuous transfer of data and communication between subject and HCP to detect clinically important changes. The data provided to the HCP enabled the HCP to identify subjects at risk of developing grade 3 or 4 CRS, NT, or other SAEs requiring hospitalization during the MDMP. The HCP directed such subjects to an appropriate medical facility for hospitalization if needed.

During screening, subjects and caregivers were trained on the health management system (HMS) 270a and assessed for compliance. Once enrolled, subjects received 2 complete cycles of blinatumomab in the outpatient setting in accordance with the monitoring and intervention guidelines. After the end of cycle 2 clinical assessment visit, some subjects may continue to receive 2 additional (optional) cycles of outpatient blinatumomab. The end of study visit occurred 30 days (3 days) after the last dose of blinatumomab 506a, 507b. The end coincided with the end of cycle 2 clinical assessment for subjects ending after cycle 2 or after cycle 3 or 4 if subjects and PI's chose to receive the optional consolidation cycles 3 and/or 4 as illustrated in FIG. 6A.

The overall study design was described by the study schema of FIG. 6A or 6B. The endpoints are defined 604a, 606a, 605b, 607b. Treatment, as defined in the example study, included any IP(s), procedure or process, non-IP(s), placebo, or medical device(s) intended to be administered to a study subject according to the study protocol. The investigational nature of this example study was in using blinatumomab in a complete outpatient setting. Both blinatumomab and the health management system (HMS) 270a are approved for use and was an approved indication. A modular Investigational Product Instruction Manual (IPIM), a document external to this protocol, contains detailed information regarding the storage, preparation, destruction, and administration of each treatment as illustrated in Table 3.

TABLE 3
Example Study Objectives and End Points

| | |
|---|---|
| Objectives | Endpoints |

Primary

| | |
|---|---|
| To determine the safety of outpatient | Incidence of grade 3 and/or |
| blinatumomab administration, during | 4 CRS, NT or any adverse |
| the MDMP (defined as the first 3 days | event (AE) requiring |
| of cycle 1 and first 2 days of cycle 2 of | hospitalization (SAE) during |
| outpatient blinatumomab infusion) | MDMP. |

Secondary

| | |
|---|---|
| To determine the time from first | Time (in minutes) from first |
| detection of grade 3 or 4 vital sign, or | onset of fever, hypotension, |
| significant clinical change to | hypoxia, other grade 3 or |
| therapeutic intervention (any | 4 vital sign including seizure or |
| measurable action taken by or | neurological change (grade |
| performed on the subject due to onset | 3-limiting self-care activities of |
| of the clinical parameters described | daily living [ADL]) to |
| above) during the MDMP | therapeutic intervention^{a} |
| Evaluate the safety and tolerability of | Overall incidence and severity |
| blinatumomab administered | of treatment-emergent |
| exclusively as an outpatient | adverse events (TEAE) and |
| | adverse events of interest in |
| | particular CRS and NT. |
| Evaluate the impact of complete | European Organisation for |
| outpatient blinatumomab treatment on | Research and Treatment of |
| patient-reported outcome (PRO), | Cancer (EORTC) validated |
| global health status (GHS), and | electronic version of |
| quality of life (QoL). | QLQ-C30. |
| Estimate healthcare resource | TEAEs that resulted in |
| utilization associated with | hospitalizations |
| treatment-related AEs | TEAEs that resulted in |
| | surgeries. |
| | TEAEs that resulted in the use |
| | of concomitant medications |
| | TEAEs that resulted in the use |
| | of device/procedure |
| | intervention. |

ADL = activities of daily living: AE = adverse event; CG = caregiver; CRS = cytokine release syndrome; EMS = Emergency Medical Service; EORTC = European Organisation for Research and Treatment of Cancer; GHS = global health status; HCP = Health Care Provider; IV = intravenous; MDMP = mandatory device monitoring period; NT = neurotoxicity; PO = by mouth; PRO = patient-reported outcomes; QLQ-30 = Quality of Life of Cancer Patients; QoL = quality of life; SAE = serious adverse event; TEAE = treatment-emergent adverse event.

Therapeutic intervention is any measurable action taken by the subject or performed on the subject as a result of the onset of the clinical parameters described. Such actions may include: Advice from HCP to immediately call EMS or dial 911, discontinuation of the blinatumomab infusion, subject taking an oral medication, subject receiving a medication or intervention by any emergency or hospital medical services, intervention such as administering any medication (IV or PO or PR) or IV fluids or oxygen etc Time to therapeutic intervention (TTI) is the measured time between the delivery of the device alert or unscheduled contact with HP by subject/CG to report a change to the time of initiation of the therapeutic intervention as defined.

TABLE 4
Example Study Treatments

| | Amgen Investigational Product:^{a} |
|---|---|
| Study Treatment Name | Blinatumomab; Blincyto ® |
| Dosage Formulation | 38.5 μg powder for solution for infusion, single use |
| | vials |
| Unit Dose Strength(s)/ | 28 μg/day as a CiVI per cycle for up to 4 cycles. |
| Dosage Level(s) and Dosage | Dose for subjects <45 kg: 15 μg/m²/day (max |
| Frequency | 28 μg/day). 1 cycle consists of 6 weeks (4 weeks |
| | CiVI and 2 weeks infusion-free interval) |
| Route of Administration | CiVI |
| Accountability | The number of vials dispensed, date dispensed, date, |
| | and lot number of investigational product (IP) are to |
| | be recorded. |
| Dosing Instructions | To prepare Blinatumomab for IV infusion, the |
| | lyophilized powder is reconstituted with sterile water |
| | for injection resulting in a concentration of |
| | 12.5 μg/mL. The reconstituted solution is added to |
| | an infusion bag containing 0.9% NaCl and a product |
| | specific stabilizer (IV Solution Stabilizer [IVSS]). The |
| | IVSS functions to prevent adsorption of |
| | blinatumomab to surfaces of the infusion |
| | components. |
| | IV infusion method: Blinatumomab infusion for |
| | solution will be prepared in IV bags for CiVI and |
| | delivered through infusion lines with an in-line 0.2 μm |
| | filter that is compatible with the IP as described in the |
| | IPIM. |
| | The daily blinatumomab dose may be up to 10% |
| | lower or higher, in order to account for possible pump |
| | inaccuracies. |

CiVI = continuous intravenous infusion; IM = intramuscular; IP = Investigational Product;
IPIM = Investigational Product Instruction Manual; IV = intravenous; IVSS = intravenous solution stabilizer
^{a}Blinatumomab will be manufactured and packaged by Amgen and distributed using Amgen clinical study drug distribution procedures.

The medical devices provided by Amgen for use in this study include: infusion pump and infusion line (e.g., infusion 110 of FIG. 1); and a health management system (HMS) 270a. These devices are non-Amgen non-investigational devices. Detail descriptions of the devices are provided herein. Blinatumomab was administered in the outpatient setting using infusion pumps approved for use by the appropriate regulatory authorities in the US. Blinatumomab solution for infusion was prepared in intravenous (IV) bags for IV infusion 110 and delivered through infusion lines that are compatible with the IP as described in the IPIM. The blinatumomab final solution for infusion should not come into contact with the pump at any time. Additional details for the use of the above-mentioned medical devices are provided in the IPIM.

In at least part, infusion pumps 110, IV bags and tubing, and ancillary materials (e.g., syringes, sterile needles, alcohol prep pads, etc.) were provided by Amgen when the site was unable to provision. Additional non-investigational medical devices that were commercially available and were not provided or reimbursed by Amgen. The investigator overseeing the conduct of the example study at each respective institution were responsible for obtaining these supplies. When site supplies were used, the Investigator was responsible for obtaining and maintaining these supplies.

A central component of the health management system (HMS) 270a was a patient wearable device 115, 215a, 315a-c (i.e., as available from Current Health, Biobeat, etc.). In any event, the patient wearable device 115, 215a, 315a-c worn on the upper arm. The wearable device 115 monitored a subject's HR, RR, skin temperature, oxygen saturation, step count and activity levels at a maximum rate of every 2 seconds. For this example study, the following vital signs were collected and evaluated: HR, RR, axillary temperature, and oxygen saturation.

The patient wearable device 115 was also integrated with the axillary temperature sensor (e.g., auxiliary temperature sensor 241m of FIG. 2M, 441a of FIG. 4A, etc.), via Bluetooth, to measure axillary temperature. In addition, a patient device (e.g., tablet device 150 of FIG. 1, 250m of FIG. 2M, etc.) allowed capture of systolic and diastolic BP via a Bluetooth integration with an approved and integrated BP monitor (e.g., blood pressure monitor 140 of FIG. 1, 240m of FIG. 2M, 441d of FIG. 4D, etc.) which the subject applied to their other arm/wrist to capture the BP. The patient wearable device 215m transmits encrypted data over WiFi in, for example, accordance with the health monitoring systems 200m,n of FIGS. 2M and 2N.

Sites administered blinatumomab as per the FDA-approved guidelines. Common Terminology Criteria for Adverse Events (CTCAE) v 5.0 will be used to grade toxicities. grade 1 and grade 2 events will be managed symptomatically with temporary discontinuation of the blinatumomab (if considered necessary by the investigator). The investigator will intervene in accordance with the alert algorithm for other AEs.

TABLE 5
Infusion Interruptions/Dose Modifications Due to Adverse Events

| | Grade or | Instructions for Treatment Interruption and Restart | |
|---|---|---|---|
| | Adverse | Subjects Greater Than or Equal | |
| Toxicity | Event | to 45 kg | Subjects Less Than 45 kg |
| Cytokine | 3 | Interrupt blinatumomab. | Interrupt blinatumomab. |
| Release | | Administer dexamethasone | Administer dexamethasone |
| Syndrome | | 8 mg every 8 hours IV or | 5 mg/m² (maximum 8 mg) |
| | | PO for up to 3 days and | every 8 hours IV or PO for |
| | | taper thereafter over | up to 3 days and taper |
| | | 4 days. | thereafter over 4 days. |
| | | When CRS is | When CRS is resolved, |
| | | resolved, restart | restart blinatumomab at |
| | | blinatumomab at | 5 μg/m²/day and escalate to |
| | | 9 μg/day, and | 15 μg/m²/day after 7 days if |
| | | escalate to 28 μg/day | the toxicity does not recur. |
| | | after 7 days if the | |
| | | toxicity does not recur. | |
| | 4 | Discontinue BLINCYTO permanently. Administer dexamethasone | |
| | | as instructed for grade 3 CRS. | |
| Neurologic | 3 | Withhold blinatumomab until no | Withhold blinatumomab until no |
| Toxicity | | more than grade 1 (mild) and for | more than grade 1 (mild) and for |
| | | at least 3 days, then restart | at least 3 days, then restart |
| | | blinatumomab at 9 μg/day. | blinatumomab at 5 μg/m²/day. |
| | | Escalate to 28 μg/day after | Escalate to 15 μg/m²/day after |
| | | 7 days if the toxicity does not | 7 days if the toxicity does not |
| | | recur. If the toxicity occurred at | recur. If the toxicity occurred at |
| | | 9 ug/day, or if the toxicity takes | 5 μg/m²/day, or if the toxicity |
| | | more than 7 days to resolve, | takes more than 7 days to |
| | | discontinue blinatumomab | resolve, discontinue |
| | | permanently. | blinatumomab permanently. |
| | 4 | Discontinue blinatumomab permanently. | |
| | Seizure^{a} | Administer dexamethasone as instructed for grade 3 CRS. | |
| | | Administer anti-seizure medication. Permanently discontinue | |
| | | BLINCYTO if seizure occurs at 9 ug or if second seizure occurs | |
| | | after restart. | |
| Other | 3 | Withhold blinatumomab until no | Withhold blinatumomab until no |
| clinically | | more than grade 1 (mild), then | more than grade 1 (mild), then |
| relevant | | restart blinatumomab at | restart blinatumomab at |
| adverse | | 9 μg/day. Escalate to 28 μg/day | 5 μg/m²/day. Escalate to |
| events | | after 7 days if the toxicity does | 15 μg/m²/day after 7 days if the |
| | | not recur. If the toxicity takes | toxicity does not recur. If the |
| | | more than 14 days to resolve, | toxicity takes more than 14 days |
| | | discontinue blinatumomab | to resolve, discontinue |
| | | permanently. | blinatumomab permanently. |

| | |
|---|---|
| 4 | Consider discontinuing blinatumomab permanently. |

^{a}Obtain brain MRI and perform cerebrospinal fluid (CSF) analysis, if indicated, and if there are no contraindications.
CRS = cytokine release syndrome; IV = intravenous(ly); PO = oral.
Grade scale is based on Common Terminology Criteria for Adverse Events version 5.0 (CTCAE v5.0).
Source: Blinatumomab USPI

Compliance with using the devices 110, 115, 140, 150, 200a, 200m, 200n was determined from the data transmitted by the device. Device alerts confirmed when data was not being transmitted from the devices (i.e., the patient wearable device 115, the auxiliary temperature monitor 241m, and the blood pressure monitor 240m). The subject was instructed to call HCP immediately when alert was received that data was not being transmitted from the devices 115, 240m, 241m. The HCP or designee were advised to contact the subject immediately if data was not being received. Assessing compliance with blinatumomab therapy per protocol and per IPIM was performed by study centers and clinical research associates. When a subject failed to comply with the instructions for use of the device, the subject received re-education/re-training on use of the device. When the subject failed to comply a second time, the subject was removed from the study and the subject was recommended for admission to a hospital for the remainder of the MDMP as per the product FDA label.

Digital health device monitoring of vital signs of patients during the mandatory monitoring period was conducted. During the MDMP, subjects wore a patient wearable device 115 continuously 24 hours a day. After the first 24 hours, subjects were allowed to remove one or all of the devices for a maximum of 1 hour per day. This allowed subjects to attend to personal activities such as taking a bath or shower. Subject was clinically stable with normal vitals and no evidence of NT prior to the start of the 60-minute period. The breaks were coordinated with the HCP, as no data was transmitted during this period. It was recommended that subjects use this time to change the device(s) and place it (them) in the charger (e.g., charger 442a of FIG. 4A). It was also strongly encouraged that the subjects change the CH wearable device (for recharging) at approximately the same time each day.

Vital signs (HR, RR, oxygen saturation, and axillary temperature) were measured continuously using health management system (HMS) 270a, as specified in a schedule of activities. There was no data transmission to HCP while subjects were transported to home from the outpatient facility. Vital sign data (i.e., sensor data) was stored but not transmitted in real time. Non-invasive BP was performed as specified in the schedule of activities. Vital signs were available as a constant live feed to the HCP or designee for the entire MDMP. Alarms/alerts were generated, and indicated when a vital sign was outside of the preset parameters. Details of the data that was monitored using the devices 115, 140 is described in detail herein.

The digital health tools used in this example study collected data beyond what was specified for analysis in this protocol. Digital Health is capable of collecting HR, RR, axillary temperature, skin temperature, oxygen saturation, step count and activity levels, and noninvasive BP. The patient device 150 also supported collection of subject responses in accordance with QLQ-C30.

The above description describes various devices, assemblies, components, subsystems and methods for use related to a drug delivery device such as a pre-filled syringe. The devices, assemblies, components, subsystems, methods or drug delivery devices (i.e., prefilled syringe) can further comprise or be used with a drug including but not limited to those drugs identified below as well as their generic and biosimilar counterparts. The term drug, as used herein, can be used interchangeably with other similar terms and can be used to refer to any type of medicament or therapeutic material including traditional and non-traditional pharmaceuticals, nutraceuticals, supplements, biologics, biologically active agents and compositions, large molecules, biosimilars, bioequivalents, therapeutic antibodies, polypeptides, proteins, small molecules and generics. Non-therapeutic injectable materials are also encompassed. The drug may be in liquid form, a lyophilized form, or in a reconstituted from lyophilized form. The following example list of drugs should not be considered as all-inclusive or limiting.

The drug will be contained in a reservoir within the pre-filled syringe for example. In some instances, the reservoir is a primary container that is either filled or pre-filled for treatment with the drug. The primary container can be a vial, a cartridge or a pre-filled syringe. In some embodiments, the reservoir of the drug delivery device may be filled with or the device can be used with colony stimulating factors, such as granulocyte colony-stimulating factor (G-CSF). Such G-CSF agents include but are not limited to Neulasta® (pegfilgrastim, pegylated filgastrim, pegylated G-CSF, pegylated hu-Met-G-CSF) and Neupogen® (filgrastim, G-CSF, hu-MetG-CSF), UDENYCA® (pegfilgrastim-cbqv), Ziextenzo® (LA-EP2006; pegfilgrastim-bmez), or FULPHILA (pegfilgrastim-bmez).

In other embodiments, the drug delivery device may contain or be used with an erythropoiesis stimulating agent (ESA), which may be in liquid or lyophilized form. An ESA is any molecule that stimulates erythropoiesis. In some embodiments, an ESA is an erythropoiesis stimulating protein. As used herein, “erythropoiesis stimulating protein” means any protein that directly or indirectly causes activation of the erythropoietin receptor, for example, by binding to and causing dimerization of the receptor. Erythropoiesis stimulating proteins include erythropoietin and variants, analogs, or derivatives thereof that bind to and activate erythropoietin receptor; antibodies that bind to erythropoietin receptor and activate the receptor; or peptides that bind to and activate erythropoietin receptor. Erythropoiesis stimulating proteins include, but are not limited to, Epogen® (epoetin alfa), Aranesp® (darbepoetin alfa), Dynepo® (epoetin delta), Mircera® (methyoxy polyethylene glycol-epoetin beta), Hematide®, MRK-2578, INS-22, Retacrit® (epoetin zeta), Neorecormon® (epoetin beta), Silapo® (epoetin zeta), Binocrit® (epoetin alfa), epoetin alfa Hexal, Abseamed® (epoetin alfa), Ratioepo® (epoetin theta), Eporatio® (epoetin theta), Biopoin® (epoetin theta), epoetin alfa, epoetin beta, epoetin iota, epoetin omega, epoetin delta, epoetin zeta, epoetin theta, and epoetin delta, pegylated erythropoietin, carbamylated erythropoietin, as well as the molecules or variants or analogs thereof.

Among particular illustrative proteins are the specific proteins set forth below, including fusions, fragments, analogs, variants or derivatives thereof: OPGL specific antibodies, peptibodies, related proteins, and the like (also referred to as RANKL specific antibodies, peptibodies and the like), including fully humanized and human OPGL specific antibodies, particularly fully humanized monoclonal antibodies; Myostatin binding proteins, peptibodies, related proteins, and the like, including myostatin specific peptibodies; IL-4 receptor specific antibodies, peptibodies, related proteins, and the like, particularly those that inhibit activities mediated by binding of IL-4 and/or IL-13 to the receptor; Interleukin 1-receptor 1 (“IL1-R1”) specific antibodies, peptibodies, related proteins, and the like; Ang2 specific antibodies, peptibodies, related proteins, and the like; NGF specific antibodies, peptibodies, related proteins, and the like; CD22 specific antibodies, peptibodies, related proteins, and the like, particularly human CD22 specific antibodies, such as but not limited to humanized and fully human antibodies, including but not limited to humanized and fully human monoclonal antibodies, particularly including but not limited to human CD22 specific IgG antibodies, such as, a dimer of a human-mouse monoclonal hLL2 gamma-chain disulfide linked to a human-mouse monoclonal hLL2 kappa-chain, for example, the human CD22 specific fully humanized antibody in Epratuzumab, CAS registry number 501423-23-0; IGF-1 receptor specific antibodies, peptibodies, and related proteins, and the like including but not limited to anti-IGF-1R antibodies; B-7 related protein 1 specific antibodies, peptibodies, related proteins and the like (“B7RP-1” and also referring to B7H2, ICOSL, B7h, and CD275), including but not limited to B7RP-specific fully human monoclonal IgG2 antibodies, including but not limited to fully human IgG2 monoclonal antibody that binds an epitope in the first immunoglobulin-like domain of B7RP-1, including but not limited to those that inhibit the interaction of B7RP-1 with its natural receptor, ICOS, on activated T cells; IL-15 specific antibodies, peptibodies, related proteins, and the like, such as, in particular, humanized monoclonal antibodies, including but not limited to HuMax IL-15 antibodies and related proteins, such as, for instance, 145c7; IFN gamma specific antibodies, peptibodies, related proteins and the like, including but not limited to human IFN gamma specific antibodies, and including but not limited to fully human anti-IFN gamma antibodies; TALL-1 specific antibodies, peptibodies, related proteins, and the like, and other TALL specific binding proteins; Parathyroid hormone (“PTH”) specific antibodies, peptibodies, related proteins, and the like; Thrombopoietin receptor (“TPO-R”) specific antibodies, peptibodies, related proteins, and the like; Hepatocyte growth factor (“HGF”) specific antibodies, peptibodies, related proteins, and the like, including those that target the HGF/SF:cMet axis (HGF/SF:c-Met), such as fully human monoclonal antibodies that neutralize hepatocyte growth factor/scatter (HGF/SF); TRAIL-R2 specific antibodies, peptibodies, related proteins and the like; Activin A specific antibodies, peptibodies, proteins, and the like; TGF-beta specific antibodies, peptibodies, related proteins, and the like; Amyloid-beta protein specific antibodies, peptibodies, related proteins, and the like; c-Kit specific antibodies, peptibodies, related proteins, and the like, including but not limited to proteins that bind c-Kit and/or other stem cell factor receptors; OX40L specific antibodies, peptibodies, related proteins, and the like, including but not limited to proteins that bind OX40L and/or other ligands of the OX40 receptor; Activase® (alteplase, tPA); Aranesp® (darbepoetin alfa) Erythropoietin [30-asparagine, 32-threonine, 87-valine, 88-asparagine, 90-threonine], Darbepoetin alfa, novel erythropoiesis stimulating protein (NESP); Epogen® (epoetin alfa, or erythropoietin); GLP-1, Avonex® (interferon beta-1a); Bexxar® (tositumomab, anti-CD22 monoclonal antibody); Betaseron® (interferon-beta); Campath® (alemtuzumab, anti-CD52 monoclonal antibody); Dynepo® (epoetin delta); Velcade® (bortezomib); MLN0002 (anti-α4ß7 mAb); MLN1202 (anti-CCR2 chemokine receptor mAb); Enbrel® (etanercept, TNF-receptor/Fc fusion protein, TNF blocker); Eprex® (epoetin alfa); Erbitux® (cetuximab, anti-EGFR/HER1/c-ErbB-1); Genotropin® (somatropin, Human Growth Hormone); Herceptin® (trastuzumab, anti-HER2/neu (erbB2) receptor mAb); Kanjinti™ (trastuzumab-anns) anti-HER2 monoclonal antibody, biosimilar to Herceptin®, or another product containing trastuzumab for the treatment of breast or gastric cancers; Humatrope® (somatropin, Human Growth Hormone); Humira® (adalimumab); Vectibix® (panitumumab), Xgeva® (denosumab), Prolia® (denosumab), Immunoglobulin G2 Human Monoclonal Antibody to RANK Ligand, Enbrel® (etanercept, TNF-receptor/Fc fusion protein, TNF blocker), Nplate® (romiplostim), rilotumumab, ganitumab, conatumumab, brodalumab, insulin in solution; Infergen® (interferon alfacon-1); Natrecor® (nesiritide; recombinant human B-type natriuretic peptide (hBNP); Kineret® (anakinra); Leukine® (sargamostim, rhuGM-CSF); LymphoCide® (epratuzumab, anti-CD22 mAb); Benlysta™ (lymphostat B, belimumab, anti-BlyS mAb); Metalyse® (tenecteplase, t-PA analog); Mircera® (methoxy polyethylene glycol-epoetin beta); Mylotarg® (gemtuzumab ozogamicin); Raptiva® (efalizumab); Cimzia® (certolizumab pegol, CDP 870); Soliris™ (eculizumab); pexelizumab (anti-C5 complement); Numax® (MEDI-524); Lucentis® (ranibizumab); Panorex® (17-1A, edrecolomab); Trabio® (lerdelimumab); TheraCim hR3 (nimotuzumab); Omnitarg (pertuzumab, 2C4); Osidem® (IDM-1); OvaRex® (B43.13); Nuvion® (visilizumab); cantuzumab mertansine (huC242-DM1); NeoRecormon® (epoetin beta); Neumega® (oprelvekin, human interleukin-11); Orthoclone OKT3® (muromonab-CD3, anti-CD3 monoclonal antibody); Procrit® (epoetin alfa); Remicade® (infliximab, anti-TNFα monoclonal antibody); Reopro® (abciximab, anti-GP lib/Ilia receptor monoclonal antibody); Actemra® (anti-IL6 Receptor mAb); Avastin® (bevacizumab), HuMax-CD4 (zanolimumab); Mvasi™ (bevacizumab-awwb); Rituxan® (rituximab, anti-CD20 mAb); Tarceva® (erlotinib); Roferon-A®-(interferon alfa-2a); Simulect® (basiliximab); Prexige® (lumiracoxib); Synagis® (palivizumab); 145c7-CHO (anti-IL15 antibody, see U.S. Pat. No. 7,153,507); Tysabri® (natalizumab, anti-α4integrin mAb); Valortim® (MDX-1303, anti-B. anthracis protective antigen mAb); ABthrax™; Xolair® (omalizumab); ETI211 (anti-MRSA mAb); IL-1 trap (the Fc portion of human IgG1 and the extracellular domains of both IL-1 receptor components (the Type I receptor and receptor accessory protein)); VEGF trap (Ig domains of VEGFR1 fused to IgG1 Fc); Zenapax® (daclizumab); Zenapax® (daclizumab, anti-IL-2Rα mAb); Zevalin® (ibritumomab tiuxetan); Zetia® (ezetimibe); Orencia® (atacicept, TACI-Ig); anti-CD80 monoclonal antibody (galiximab); anti-CD23 mAb (lumiliximab); BR2-Fc (huBR3/huFc fusion protein, soluble BAFF antagonist); CNTO 148 (golimumab, anti-TNFα mAb); HGS-ETR1 (mapatumumab; human anti-TRAIL Receptor-1 mAb); HuMax-CD20 (ocrelizumab, anti-CD20 human mAb); HuMax-EGFR (zalutumumab); M200 (volociximab, anti-α5β1 integrin mAb); MDX-010 (ipilimumab, anti-CTLA-4 mAb and VEGFR-1 (IMC-18F1); anti-BR3 mAb; anti-C. difficile Toxin A and Toxin B C mAbs MDX-066 (CDA-1) and MDX-1388); anti-CD22 dsFv-PE38 conjugates (CAT-3888 and CAT-8015); anti-CD25 mAb (HuMax-TAC); anti-CD3 mAb (NI-0401); adecatumumab; anti-CD30 mAb (MDX-060); MDX-1333 (anti-IFNAR); anti-CD38 mAb (HuMax CD38); anti-CD40L mAb; anti-Cripto mAb; anti-CTGF Idiopathic Pulmonary Fibrosis Phase I Fibrogen (FG-3019); anti-CTLA4 mAb; anti-eotaxin1 mAb (CAT-213); anti-FGF8 mAb; anti-ganglioside GD2 mAb; anti-ganglioside GM2 mAb; anti-GDF-8 human mAb (MYO-029); anti-GM-CSF Receptor mAb (CAM-3001); anti-HepC mAb (HuMax HepC); anti-IFNα mAb (MEDI-545, MDX-198); anti-IGF1R mAb; anti-IGF-1R mAb (HuMax-Inflam); anti-IL12 mAb (ABT-874); anti-IL12/IL23 mAb (CNTO 1275); anti-IL13 mAb (CAT-354); anti-IL2Rα mAb (HuMax-TAC); anti-IL5 Receptor mAb; anti-integrin receptors mAb (MDX-018, CNTO 95); anti-IP10 Ulcerative Colitis mAb (MDX-1100); BMS-66513; anti-Mannose Receptor/hCGβ mAb (MDX-1307); anti-mesothelin dsFv-PE38 conjugate (CAT-5001); anti-PD1mAb (MDX-1106 (ONO-4538)); anti-PDGFRα antibody (IMC-3G3); anti-TGFß mAb (GC-1008); anti-TRAIL Receptor-2 human mAb (HGS-ETR2); anti-TWEAK mAb; anti-VEGFR/Flt-1 mAb; and anti-ZP3 mAb (HuMax-ZP3).

In some embodiments, the drug delivery device may contain or be used with a sclerostin antibody, such as but not limited to romosozumab, blosozumab, BPS 804 (Novartis), Evenity™ (romosozumab-aqqg), another product containing romosozumab for treatment of postmenopausal osteoporosis and/or fracture healing and in other embodiments, a monoclonal antibody (IgG) that binds human Proprotein Convertase Subtilisin/Kexin Type 9 (PCSK9). Such PCSK9 specific antibodies include, but are not limited to, Repatha® (evolocumab) and Praluent® (alirocumab). In other embodiments, the drug delivery device may contain or be used with rilotumumab, bixalomer, trebananib, ganitumab, conatumumab, motesanib diphosphate, brodalumab, vidupiprant or panitumumab. In some embodiments, the reservoir of the drug delivery device may be filled with or the device can be used with IMLYGIC® (talimogene laherparepvec) or another oncolytic HSV for the treatment of melanoma or other cancers including but are not limited to OncoVEXGALV/CD; OrienX010; G207, 1716; NV1020; NV12023; NV1034; and NV1042. In some embodiments, the drug delivery device may contain or be used with endogenous tissue inhibitors of metalloproteinases (TIMPs) such as but not limited to TIMP-3. In some embodiments, the drug delivery device may contain or be used with Aimovig® (erenumab-aooe), anti-human CGRP-R (calcitonin gene-related peptide type 1 receptor) or another product containing erenumab for the treatment of migraine headaches. Antagonistic antibodies for human calcitonin gene-related peptide (CGRP) receptor such as but not limited to erenumab and bispecific antibody molecules that target the CGRP receptor and other headache targets may also be delivered with a drug delivery device of the present disclosure.

Additionally, the sensors, devices, systems and methods of the present disclosure may be particularly advantageous in regard to out-patient treatments that may be associated with particular adverse side effects caused by similar molecules having a specific mechanism of action (MOA). For example, a bispecific T cell engager (BiTE®) molecules such as, but not limited to, BLINCYTO® (blinatumomab) can be used in or with the drug delivery device of the present disclosure. In some embodiments, the drug delivery device may contain or be used with AMG 160, or another product that contains a half-life extended (HLE) anti-prostate-specific membrane antigen (PSMA)×anti-CD3 BiTE® (bispecific T cell engager) construct. In some embodiments, the drug delivery device may contain or be used with AMG 199 or another product containing a half-life extended (HLE) bispecific T cell engager construct (BiTE®). In some embodiments, the drug delivery device may contain or be used with AMG 330 or another product containing an anti-CD33×anti-CD3 BiTE® (bispecific T cell engager) construct. In some embodiments, the drug delivery device may contain or be used with AMG 427 or another product containing a half-life extended (HLE) anti-fms-like tyrosine kinase 3 (FLT3)×anti-CD3 BiTE® (bispecific T cell engager) construct. In some embodiments, the drug delivery device may contain or be used with AMG 509 or another product containing a bivalent T-cell engager and is designed using XmAb® 2+1 technology. In some embodiments, the drug delivery device may contain or be used with AMG 562 or another product containing a half-life extended (HLE) CD19×CD3 BiTE® (bispecific T cell engager) construct. In some embodiments, the drug delivery device may contain or be used with AMG 596 or another product containing a CD3×epidermal growth factor receptor vIII (EGFRvIII) BiTE® (bispecific T cell engager) molecule. In some embodiments, the drug delivery device may contain or be used with AMG 673 or another product containing a half-life extended (HLE) anti-CD33×anti-CD3 BiTE® (bispecific T cell engager) construct. In some embodiments, the drug delivery device may contain or be used with AMG 701 or another product containing a half-life extended (HLE) anti-B-cell maturation antigen (BCMA)×anti-CD3 BiTE® (bispecific T cell engager) construct. In some embodiments, the drug delivery device may contain or be used with AMG 757 or another product containing a half-life extended (HLE) anti-delta-like ligand 3 (DLL3)×anti-CD3 BiTE® (bispecific T cell engager) construct. In some embodiments, the drug delivery device may contain or be used with AMG 910 or another product containing a half-life extended (HLE) epithelial cell tight junction protein claudin 18.2×CD3 BiTE® (bispecific T cell engager) construct.

In some embodiments, the drug delivery device may contain or be used with an APJ large molecule agonist such as but not limited to apelin or analogues thereof. In some embodiments, a therapeutically effective amount of an anti-thymic stromal lymphopoietin (TSLP) or TSLP receptor antibody is used in or with the drug delivery device of the present disclosure. In some embodiments, the drug delivery device may contain or be used with Avsola™ (infliximab-axxq), anti-TNF a monoclonal antibody, biosimilar to Remicade® (infliximab) (Janssen Biotech, Inc.) or another product containing infliximab for the treatment of autoimmune diseases. In some embodiments, the drug delivery device may contain or be used with Kyprolis® (carfilzomib), (2S)-N-((S)-1-((S)-4-methyl-1-((R)-2-methyloxiran-2-yl)-1-oxopentan-2-ylcarbamoyl)-2-phenylethyl)-2-((S)-2-(2-morpholinoacetamido)-4-phenylbutanamido)-4-methylpentanamide, or another product containing carfilzomib for the treatment of multiple myeloma. In some embodiments, the drug delivery device may contain or be used with Otezla® (apremilast), N-[2-[(15)-1-(3-ethoxy-4-methoxyphenyl)-2-(methylsulfonyl)ethyl]-2,3-dihydro-1,3-dioxo-1H-isoindol-4-yl]acetamide, or another product containing apremilast for the treatment of various inflammatory diseases. In some embodiments, the drug delivery device may contain or be used with Parsabiv™ (etelcalcetide HCl, KAI-4169) or another product containing etelcalcetide HCl for the treatment of secondary hyperparathyroidism (sHPT) such as in patients with chronic kidney disease (KD) on hemodialysis. In some embodiments, the drug delivery device may contain or be used with ABP 798 (rituximab), a biosimilar candidate to Rituxan®/MabThera™, or another product containing an anti-CD20 monoclonal antibody. In some embodiments, the drug delivery device may contain or be used with a VEGF antagonist such as a non-antibody VEGF antagonist and/or a VEGF-Trap such as aflibercept (Ig domain 2 from VEGFR1 and Ig domain 3 from VEGFR2, fused to Fc domain of IgG1). In some embodiments, the drug delivery device may contain or be used with ABP 959 (eculizumab), a biosimilar candidate to Soliris®, or another product containing a monoclonal antibody that specifically binds to the complement protein C5. In some embodiments, the drug delivery device may contain or be used with Rozibafusp alfa (formerly AMG 570) is a novel bispecific antibody-peptide conjugate that simultaneously blocks ICOSL and BAFF activity. In some embodiments, the drug delivery device may contain or be used with Omecamtiv mecarbil, a small molecule selective cardiac myosin activator, or myotrope, which directly targets the contractile mechanisms of the heart, or another product containing a small molecule selective cardiac myosin activator. In some embodiments, the drug delivery device may contain or be used with Sotorasib (formerly known as AMG 510), a KRAS^{G12C} small molecule inhibitor, or another product containing a KRAS^{G12C} small molecule inhibitor. In some embodiments, the drug delivery device may contain or be used with Tezepelumab, a human monoclonal antibody that inhibits the action of thymic stromal lymphopoietin (TSLP), or another product containing a human monoclonal antibody that inhibits the action of TSLP. In some embodiments, the drug delivery device may contain or be used with AMG 714, a human monoclonal antibody that binds to Interleukin-15 (IL-15) or another product containing a human monoclonal antibody that binds to Interleukin-15 (IL-15). In some embodiments, the drug delivery device may contain or be used with AMG 890, a small interfering RNA (siRNA) that lowers lipoprotein(a), also known as Lp(a), or another product containing a small interfering RNA (siRNA) that lowers lipoprotein(a). In some embodiments, the drug delivery device may contain or be used with ABP 654 (human IgG1 kappa antibody), a biosimilar candidate to Stelara®, or another product that contains human IgG1 kappa antibody and/or binds to the p40 subunit of human cytokines interleukin (IL)-12 and IL-23. In some embodiments, the drug delivery device may contain or be used with Amjevita™ or Amgevita™ (formerly ABP 501) (mab anti-TNF human IgG1), a biosimilar candidate to Humira®, or another product that contains human mab anti-TNF human IgG1. In some embodiments, the drug delivery device may contain or be used with AMG 119, or another product containing a delta-like ligand 3 (DLL3) CART (chimeric antigen receptor T cell) cellular therapy. In some embodiments, the drug delivery device may contain or be used with AMG 119, or another product containing a delta-like ligand 3 (DLL3) CAR T (chimeric antigen receptor T cell) cellular therapy. In some embodiments, the drug delivery device may contain or be used with AMG 133, or another product containing a gastric inhibitory polypeptide receptor (GIPR) antagonist and GLP-1R agonist. In some embodiments, the drug delivery device may contain or be used with AMG 171 or another product containing a Growth Differential Factor 15 (GDF15) analog. In some embodiments, the drug delivery device may contain or be used with AMG 176 or another product containing a small molecule inhibitor of myeloid cell leukemia 1 (MCL-1). In some embodiments, the drug delivery device may contain or be used with AMG 256 or another product containing an anti-PD-1×IL21 mutein and/or an IL-21 receptor agonist designed to selectively turn on the Interleukin 21 (IL-21) pathway in programmed cell death-1 (PD-1) positive cells. In some embodiments, the drug delivery device may contain or be used with AMG 404 or another product containing a human anti-programmed cell death-1 (PD-1) monoclonal antibody being investigated as a treatment for patients with solid tumors. In some embodiments, the drug delivery device may contain or be used with AMG 430 or another product containing an anti-Jagged-1 monoclonal antibody. In some embodiments, the drug delivery device may contain or be used with AMG 506 or another product containing a multi-specific FAP×4-1BB-targeting DARPin® biologic under investigation as a treatment for solid tumors. In some embodiments, the drug delivery device may contain or be used with Efavaleukin alfa (formerly AMG 592) or another product containing an IL-2 mutein Fc fusion protein.

Although the drug delivery devices, assemblies, components, subsystems and methods have been described in terms of exemplary embodiments, they are not limited thereto. The detailed description is to be construed as exemplary only and does not describe every possible embodiment of the present disclosure. Numerous alternative embodiments could be implemented, using either current technology or technology developed after the filing date of this patent that would still fall within the scope of the claims defining the invention(s) disclosed herein.

Those skilled in the art will recognize that a wide variety of modifications, alterations, and combinations can be made with respect to the above described embodiments without departing from the spirit and scope of the invention(s) disclosed herein, and that such modifications, alterations, and combinations are to be viewed as being within the ambit of the inventive concept(s).

1. A system for out-patient treatment, the system comprising:
an infusion pump for delivering a therapeutic to a patient;
a wearable device configured to be worn by the patient at least one of: (a) before delivery of the therapeutic and (b) during delivery of the therapeutic, the wearable device comprising one or more sensors configured to acquire sensor data related to detection of patient vital signs; and
a wireless communication module disposed on the wearable device and configured for remote wireless communication with a healthcare provider based on the acquired sensor data.

2. The system of claim 1, wherein the infusion pump comprises the therapeutic.

3. The system of claim 1, wherein the therapeutic is known to trigger an immune inflammatory response in the patient, potentially leading to an increased risk factors of (a) cytokine release syndrome (CRS) or (b) neurotoxicity (NT).

4. (canceled)

5. The system of claim 1, wherein the therapeutic comprises at least one of (a) an immunotherapy drug that activates patient T cells to fight cancer, (b) an immunotherapy drug including blinatumomab, and (c) an immunotherapy drug including acepatamab.

6. (canceled)

7. (canceled)

8. The system of claim 1, wherein the at least one sensor of the wearable device comprises an optical sensor that is configured to measure oxygen of the patient.

9. The system of claim 1, wherein the at least one sensor of the wearable device is configured to measure at least one indicator of cytokine release syndrome selected from: (a) heart rate (HR), (b) axillary temperature, (c) respiratory rate (RR), (d) oxygen saturation, and (e) blood pressure (BP).

10. The system of claim 1, wherein the at least one sensor continuously measures at least one of: (a) heart rate, (b) temperature, (c) respiratory rate, and (d) oxygen saturation.

11. The system of claim 1, wherein the wireless communication module of the wearable device is configured to at least one of (a) send messages to the healthcare provider in real time, (b) send periodic messages to the healthcare provider based on an interval of a predetermined number of second, minutes, or hours, (c) send periodic messages every ten minutes, (d) send an alarm message when oxygen of the patient drops below a predetermined threshold, and (e) send an alarm if the communication is disrupted.

12. (canceled)

13. (canceled)

14. (canceled)

15. (canceled)

16. The system of claim 1, further comprising an axillary temperature sensor for detecting a temperature of the patient during delivery of the therapeutic, the axillary temperature sensor communicatively coupled to the wearable device.

17. The system of claim 16, where in the axillary temperature sensor includes a wireless communication module configured to communicate with the wireless communication module of the wearable device.

18. The system of claim 1, further comprising a blood pressure cuff for detecting a blood pressure of the patient during delivery of the therapeutic, wherein the blood pressure cuff is communicatively coupled to the wearable device.

19. The system of claim 1, wherein the healthcare provider comprises a mobile device configured for wireless communication with the wireless communication module of the wearable device.

20. The system of claim 19, further comprising a patient-assigned wireless device comprising a mobile application configured to support audio and/or video communication with the mobile device of the healthcare provider.

21. A computer-implemented method for out-patient treatment with immunotherapy that activates T cells of a patient to kill cancer, the method comprising:
receiving, at a processor, sensor data in response to the processor executing a sensor data receiving module; and
generating, using a processor, warning data, based on the sensor data, in response to the processor executing a warning data generation module, wherein a content of the sensor data is based on a likelihood that the immunotherapy will trigger increased risk factors of cytokine release syndrome.

22. The computer-implemented method as in claim 21, further comprising:
transmitting, using a processor, the sensor data to at least one of (a) a healthcare provider device in response to the processor executing a sensor data transmission module, and (b) to a patient device, wherein the sensor data is representative of vital signs of the patient.

23. The computer-implemented method of claim 21, further comprising:
transmitting, using a processor, the warning data to a healthcare provider device (a) in response to the processor executing a warning data transmission module or (b) when the sensor data exceeds a threshold value, in response to the processor executing a warning data transmission module.

24. The computer-implemented method of claim 21, further comprising:
transmitting, using a processor, audio/video data between a patient device and a healthcare provider smartphone via a cellular connection in response to the processor executing an audio/video data transmission module.

25. (canceled)

26. The computer-implemented method of claim 21, further comprising:
transmitting, using a processor, patient-initiated call input data to at least one of: a healthcare provider, or a health management system, in response to the processor executing a patient-initiated call input receiving module.

27. The computer-implemented method as in claim 26, wherein a healthcare provider phone is configured to emit a loud audible alarm, based on the warning data, at a periodic interval until a health care provider responds.

28. (canceled)

29. The computer-implemented method of claim 21, wherein the sensor data is representative of at least one indicator of cytokine release syndrome selected from: heart rate (HR), axillary temperature, respiratory rate (RR), oxygen saturation, and blood pressure (BP).
